(19) United States
(12) Patent Application Publication
(10) Pub. No.: US 2003/0199092 A1
(43) Pub. Date: Oct. 23, 2003

(54) Methods for increasing mRNA half-life in eukaryotic cells

(75) Inventor: David L. Meyer, Los Angeles, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/342,136

(22) Filed: Jan. 13, 2003

Related U.S. Application Data
(60) Provisional application No. 60/347,533, filed on Jan. 11, 2002.

(51) Int. Cl. A61K 48/00; C12N 15/85

(52) U.S. Cl. 435/455; 514/44

(57) ABSTRACT

A method is provided whereby altered cells exhibit increased intracellular half-life of transcribed mRNAs resulting in increased levels of expressed and/or secreted proteins. The cells are genetically altered to increase the level of intracellular ribosome receptor, which induces mRNA half-life.

### CROSS-REFERENCE TO RELATED APPLICATIONS

This application claims priority to and benefit of U.S. S No. 60/347,533, filed on Jan. 11, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported by Grant No: GM38538 and Grant No: GM55052 from the National Institutes of Health. The Government of the United States of America may have certain rights in this invention.

### FIELD OF THE INVENTION

The present invention generally relates to methods for increasing the resident half-life of mRNAs thereby resulting in improved expression and/or secretion of expressed proteins.

### BACKGROUND OF THE INVENTION

An objective of many biotechnology and pharmaceutical companies is the production of large quantities of recombinant proteins that can be used in a variety of industrial, agricultural, medical, and research applications. Two examples receiving significant publicity in recent years are TPA (tissue plasminogen activator) and EPO (erythropoeitin).

Methods have been sought to increase the secretion of recombinant proteins from the cell culture systems being used to produce them. For example, as noted in U.S. Pat. No. 5,272,064, use of the more highly evolved eukaryotic host cell systems and yeast host cell systems for the recombinant production of platelet-derived growth factor B had typically resulted in the secretion of biologically active rPDGF B in relatively low levels and thus the inventor turned to an E. coli expression system.

Other examples are provided by U.S. Pat. No. 5,759,810, in which inventors Honjo et al. disclose a method for secreting a human growth hormone in an E. coli host cell by expressing a genetically engineered E. coli host cell to produce enhanced amounts of glutathione reductase with the target recombinant protein and U.S. Pat. No. 5,679,543 in which inventor Lawlis, describes fusion DNA sequences that, when expressed in a filamentous fungus, are said to result in increased levels of secretion of the desired polypeptide, such as human tissue plasminogen activator, human growth hormone, human interferon, etc.

Although these aforementioned technologies may have been advantageous for secreting a number of proteins, these expression systems may exhibit several potential drawbacks. The ability to secrete proteins from both prokaryotes and eukaryotes is dependent upon the presence of signal sequences. Although these can be added to non-secreted proteins or swapped between secreted proteins, evolution has likely selected the best signal sequence to match the mature protein being secreted. In other words, splicing a signal sequence onto a protein may enable its secretion, but may have little positive effect on the rate or amount secreted. More importantly, these technologies often do not address the ability of the host cells to perform a number of covalent modifications or processing steps often so important in producing biologically active recombinant proteins. To this end, the ability to develop host cells in which the totality of the secretory apparatus—including all of the processing enzymes—is upregulated would have the greatest advantage.

In many instances, the most economical cell culture systems (bacteria, yeast, insect cells, etc.) do not produce proteins that possess adequate biological activity. In these situations, use of mammalian cells such as CHO (Chinese Hamster Ovary) is often preferred. Such mammalian cell systems, however, are more expensive due to the limited number of cells that can be grown per liter of medium, and the expensive nature of the components of the growth medium itself

Thus, a clear benefit to companies that produce their proteins in cell cultures would be the ability of cultured mammalian cells to exhibit increased levels of secretion for the recombinant proteins being produced. But even with other types of eukaryotic cells, increased protein secretion is desirable. For example, yeast has been an attractive alternative to recombinant protein production in cells other than E. coli.

### SUMMARY OF THE INVENTION

This invention pertains to the discovery that increasing the level of a ribosome receptor in a cell (e.g. p180) can increase the half-life of mRNA in that cell, particularly mRNAs that are or that become associated with a secretory pathway. Modification of an mRNA (that is normally translated in the cytosol) to enable its targeting to the ER (e.g., by incorporation of a secretory signal) caused an increase in its stability in the presence of a ribosome receptor. Membrane fractionation demonstrated that an increased stability of mRNAs targeted to the secretory pathway could be correlated with their increased association with the ER membrane in ribosome receptor-expressing cells.

Thus, in certain embodiments, this invention provides methods of increasing the intracellular half-life of an mRNA in a eukaryotic cell. The methods involve increasing the intracellular level of a ribosome receptor, as compared to the level of the ribosome receptor in an unmodified or untreated eukaryotic cell of the same type, whereby the increase in intracellular level of the ribosome receptor results in an increase in the half-life of the mRNA. The mRNA can be one that encodes a heterologous protein or an endogenous protein. In certain embodiments, the mRNA additionally encodes a protein comprising a secretory signal. In certain embodiments, the mRNA encodes a membrane bound protein. In certain embodiments, the mRNA encodes a secreted protein. The mRNA can be encoded by a nucleic acid construct that is not integrated into the genome of said cell or by a nucleic acid construct that is integrated into the genome of said cell (e.g. via homologous recombination). The mRNA can be under the control (i.e. operably linked to) a promoter (e.g. a constitutive, inducible, or tissue specific promoter) that is heterologous or endogenous to the cell. Virtually any eukaryotic cell can be used for practice of the methods of this invention. Such cells include, but are not limited to a fungal cell, a plant cell, a vertebrate cell, a protozoan cell, and an invertebrate cell. Certain preferred cells include, but are not limited to a fungal cell, a plant cell, an insect cell, and a mammalian cell (e.g. a human cell). In certain embodiments the cell is a CHO cell. In certain embodiments, the ribosome is preferably a vertebrate ribosome receptor, more preferably a mammalian ribosome receptor (e.g. a ribosome receptor from canus, equine, human, non-human primates, feline, bovine, ungulate, porcine, murine, porcine, and the like).

The intracellular level of the ribosome receptor can be increased by any of a number of convenient methods. Such methods include, but are not limited to modifying or replacing the endogenous promoter regulating expression of an endogenous ribosome receptor, or transfecting the cell with a nucleic acid construct that encodes and expresses the ribosome receptor, receptor fragment, receptor mutant, or receptor isoform. Where the cell is transfected with a construct expressing an effective ribosome receptor, in certain embodiments, the transfection is stable, while in certain other embodiments ,the transfection is transient, but of sufficient duration to increase proliferation of RER and/or to increase mRNA half-life. The construct encoding a effective ribosome receptor can comprise a plurality of regions each encoding an effective ribosome receptor. In certain embodiments, the construct encodes a mammalian ribosome receptor, preferably a full-length mammalian ribosome receptor.

In another embodiment, this invention provides methods of increasing production of a protein by a eukaryotic cell. The methods involve increasing the intracellular half-life of an mRNA encoding the protein by increasing the intracellular level of a ribosome receptor in the cell, as compared to the level of the ribosome receptor in an unmodified or untreated eukaryotic cell of the same type, whereby the increase in intracellular level of the ribosome receptor results in an increase in the half-life of the mRNA and resulting in an increase in expression of the protein. The mRNA can be one that encodes a heterologous protein or an endogenous protein. In certain embodiments, the mRNA additionally encodes a protein comprising a secretory signal. In certain embodiments, the mRNA encodes a membrane bound protein. In certain embodiments, the mRNA encodes a secreted protein. The mRNA can be encoded by a nucleic acid construct that is not integrated into the genome of said cell or by a nucleic acid construct that is integrated into the genome of said cell (e.g. via homologous recombination). The mRNA can be under the control (i.e. operably linked to) a promoter (e.g. a constitutive, inducible, or tissue specific promoter) that is heterologous or endogenous to the cell. Virtually any eukaryotic cell can be used for practice of the methods of this invention. Such cells include, but are not limited to a fungal cell, a plant cell, a vertebrate cell, a protozoan cell, and an invertebrate cell. Certain preferred cells include, but are not limited to a fungal cell, a plant cell, an insect cell, and a mammalian cell (e.g. a human cell). In certain embodiments the cell is a CHO cell. In certain embodiments, the ribosome is preferably a vertebrate ribosome receptor, more preferably a mammalian ribosome receptor (e.g. a ribosome receptor from canus, equine, human, non-human primates, feline, bovine, ungulate, porcine, murine, porcine, and the like).

The intracellular level of the ribosome receptor can be increased by any of a number of convenient methods, e.g. as described herein. The method can further involve maintaining the cell under cell culture conditions to secrete the recombinant protein therefrom. The method can also further involve purifying the protein secreted from the cell.

In still another embodiment, this invention provides kits for practice of the methods of this invention. In one embodiment the kit is a kit for increasing the half-life of an mRNA in a eukaryotic cell where the kit comprises a container containing a nucleic acid construct encoding one or more ribosome receptors; and instructional materials teaching that increasing the intracellular level of a ribosome receptor will increase the intracellular half-life of an mRNA. In another embodiment the kit is a kit for increasing the half-life of an mRNA in a eukaryotic cell, where the kit comprises a container containing a eukaryotic cell that has been treated or modified to express an elevated level of ribosome receptor as compared to an untreated or unmodified cell of the same type; and instructional materials teaching that increasing the intracellular level of a ribosome receptor will increase the intracellular half-life of an mRNA. The kits can further include a vector for introducing a nucleic acid encoding said mRNA into the cell, and/or a transfection reagent for introducing the nucleic acid construct into the cell.

This invention also pertains to the use of expression of a ribosome receptor, and parts thereof, to induce membrane proliferation in various eukaryotic cells. The membranes that have been proliferated are those needed for protein secretion in the cell types. We have demonstrated that membrane induction results in an increased secretory capacity in these cells. Thus, another aspect of the present invention pertains to a method for increasing the intracellular transport or secretion of a desired protein in a eukaryotic host cell. The method comprises expressing a gene encoding the desired protein in a eukaryotic host cell that has been genetically altered. The genetic alteration is effective to express an increased amount of at least a portion of a ribosome receptor so as to increase either the intracellular transport (if the desired protein is a membrane-bound receptor or enzyme) or the secretion of the desired protein with respect to wild-type eukaryotic host cells.

Among applications contemplated for use of the inventive cells having the genetically engineered proliferation of ribosome receptors or fragments thereof (that is, of cells exhibiting increased secretory capacity) are for the production of proteins, such as proteins useful for therapeutic purposes. Yields of the desired proteins—usually, but not always recombinant proteins—can be increased through this technology. Thus, the invention provides a process to increase the secretory capacities of cells. In this way cells can be created with an increased ability to transport or secrete a recombinant protein of interest, or, cells already expressing a desired protein can be transformed by practice of the invention to reach previously unattainable levels of transport and secretion.

Definitions

The term “ribosome receptor”, as used herein, includes full length ribosome receptors and/or ribosome receptor fragments of a length long enough to produce incr4ease in mRNA half-life, and/or proliferation of rough endoplasmic reticulum when expression level is increased in cell. Also included are ribosome receptor mutants and/or isoforms also capable of inducing an increase in mRNA half-life and/or proliferation of rough endoplasmic reticulum when expressed or overexpressed in a cell.

The term “effective ribosome receptor” refers to a ribosome receptor, a fragment or mutant thereof, and/or a ribosome receptor isoforms whose elevated expression in a cell results in increased mRNA half-life and/or increased proliferation of rough endoplasmic reticulum as compared to the same cell type that is not modified or treated to produce and elevated ribosome receptor level.

The level of a ribosome receptor in a cell treated or modified to have an increased level of ribosome receptor is said to be increased when the amount of ribosome receptor is detectably greater than the amount of ribosome receptor in the same type of cell that is untreated or unmodified or in the same cell before treatment or modification. In preferred embodiments, the increase is a statistically significant increase, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the increase is at least a 10% increase, preferably at least a 20% increase, more preferably at least a 50% increase, and most preferably at least a 90% increase.

### BRIEF DESCRIPTION OF THE DRAWINGS

FIG. 1A shows the primary structure (amino acid sequence) of the canine ribosome receptor. FIG. 1B shows a diagrammatic representation of constructs used in the examples described herein. FIG. 1C illustrates the alignment of amino acid sequences deduced from cDNA clones encoding ribosome receptor homologues from murine and human sources (dashes indicate identities, letters in mouse and human indicate variance from the canine, dots indicate putative deletions).

FIG. 2 is an electron micrograph of a vector-only control. Vector-only control yeast cells have few intracellular membranes. Cells were transformed with pYEX-BX. Transformants were selected and grown in Cu₂₁-containing medium for 5 h before preparation for electron microscopy. Visible are mitochondria, the nucleus, the vacuole, and substantial quantities of free ribosomes in the cytosol. Bars, 200 nm.

FIG. 3 is an electron micrograph of yeast showing rough membranes that are elaborated in response to the expression of the full-length ribosome receptor. Cells were transformed with pYEX-BX containing full-length p180 cDNA. Transformants were selected and grown in Cu₂₁-containing medium for 5 h before preparation for electron microscopy. Visible are extensive arrays of ribosome-studded membranes. Bars, 200 nm.

FIG. 4 is an electron micrograph of yeast expressing a construct (ΔNT in FIG. 1B) in which the ribosome binding domain has been deleted from the receptor which resulted in a proliferation of smooth membranes. Expression of p180 without its ribosome binding domain produces extensive smooth membrane proliferation different from karmellae. Cells were transfected with pYEX-BX containing the ANT version of p180. Transformants were selected and grown in Cu21-containing medium for 5 h before preparation for electron microscopy. Visible are parallel arrays of smooth membranes. Note the exclusion of cytoplasmic ribosomes from the areas where membranes are evident, and their high density in membrane free zones. Bars, 200 nm.

FIG. 5 is an electron micrograph of yeast expressing a construct that contains only the ribosome binding domain and the membrane anchor (ΔCT in FIG. 1B) that resulted in the most dramatic a proliferation of rough membranes. Expression of the NH₂-terminal half of p180 induces extensive, closely spaced rough membranes. Cells were transfected with pYEX-BX containing the ΔCT version of p180. Transformants were selected and grown in Cu21-containing medium for 5 h before preparation for electron microscopy. Visible are closely opposed rough membranes with a lower density of ribosomes in membrane-free zones. Bars, 200 nm.

FIG. 6 is a Northern blot showing that stimulation of membrane proliferation induces the expression of genes encoding proteins specific to organelles along the secretory pathway, where the gene that has been transfected into each strain is indicated at the top of the figure, and each row represents the expression levels of a gene that encodes a protein known to participate in different organelle-specific aspects of the secretory process

FIG. 7 is an indirect immunofluorescence of control yeast (top row) as well as cells expressing the ACT construct (bottom row), and shown is staining with an anti-ribosome receptor antibody (left panels), an antibody against the resident ER protein Sec6 I p (middle panels), and an antibody against Gda I p, a resident protein of the Golgi complex (right panels).

FIG. 8 is a growth assay demonstrating that cells expressing high levels of a gene encoding bovine pancreatic trypsin inhibitor (BPTI) are unable to grow unless transfected with genes encoding ACT or full-length ribosome receptor

FIG. 9 is an assay whereby levels of BPTI are measured in the culture medium of control yeast cells or of cells expressing ACT.

FIG. 10 is an electron micrograph of control monkey COS-7 cells (Panel A, lower cell, and Panel C and COS-7 cells transiently transfected with a plasmid encoding the full-length ribosome receptor (Panel A, upper cell, and Panel B)

FIG. 11 shows immunofluorescent detection of the ribosome receptor in Chinese hamster ovary (CHO) cells that have been stably transfected with cDNA encoding the ribosome receptor.

FIG. 12 shows immunofluorescent detection of the ribosome receptor in Chinese hamster ovary (CHO) cells that have been stably transfected with a cDNA construct that enables inducible expression of the ribosome receptor

FIG. 13 illustrates results of an assay for secreted alkaline phosphatase where cells described in FIG. 12 were transiently transfected with a plasmid encoding a secreted form of the enzyme alkaline phosphatase.

FIGS. 14A and 14B show that disruption of the IRE1 gene does not alter p180-induced membrane proliferations. FIG. 14A: P180 constructs used in this study. Full-length p180 contains an amino-terminal membrane-anchoring domain (black bar), a ribosome-binding domain (hashed box), and a carboxy-terminal domain of unknown function (white box). The membrane-anchor, ΔCT (lacking the C-terminal domain), and ΔNT (lacking the ribosome-binding domain) constructs are also diagramed. FIG. 14B: Δire1 cells transformed with vector or various p180 constructs were grown for 5 hours in selective media in the presence of 0.5 mM CuSO₄ before preparation for electron microscopy. Δire1 cells transformed with the empty pYEX-BX vector have few intracellular membranes; bar, 500 nm. Δire1 cells transformed with pYEX-BX containing full-length p180 coding sequence give rise to rough membrane proliferations with wide (80-100 nm) spacing; bar, 1 μm. Δire1 cells expressing pYEX-BX containing the ACT construct result in the proliferation of rough membranes with close spacing; bar, 1 μm. Δire1 cells transformed with pYEX-BX containing a sequence encoding ΔNT have smooth membrane proliferations with wide (80-100 nm) spacing; bar, 500 nm Δire1 cells transformed with pYEX-BX containing the membrane anchoring region only have smooth membrane proliferations with close spacing; bar, 500 nm.

FIG. 15 shows that increases in the steady-state levels secretory pathway mRNAs upon p180 induction occur independently of IRE1. Northern blot analysis was performed on RNA isolated from wild-type yeast and yeast in which the IRE1 gene had been disrupted. Yeast cells were transformed with the pYEX-BX vector or with pYEX-BX containing the coding sequence for a C-terminal deletion of p180 (ACT). Strains were grown for 6 hours in selective media in the presence of 0.5 mMCuSO₄. Tunicamycin (Tm) was added to one vector-control culture to 2 μg/ml for 2 hours before harvesting of cells and isolation of RNA. Northern blot analyses are shown for SEC61, KAR2, and INO1 mRNA. Values for induction are normalized to the PGK1 mRNA, whose levels remain unchanged in the presence of p180.

FIG. 16 shows that expression of ACT does not increase activity at the UPRE. β-galactosidase activity was measured in extracts of BY4733 cells harboring the UPRE-LacZ plasmid, pMCZ-Y and pYEX-BX or pYEX-BX containing ACT. Cells were grown for 5 hours in selective media with 0.5 mM CuSO₄ in the presence or absence of tunicamycin (+Tm).

FIGS. 17A through 17D show that an increased level of SEC61 mRNA is not regulated transcriptionally upon p180 induction. Effect of ACT expression on SEC61 promoter activity: β-galactosidase activity was measured in extracts of SEY6210 cells harboring a SEC61-LacZ construct in pRS314 and pYEX-BX vector or ΔCT. Cells were grown for 5 hours in selective media in the presence of 0.5 mM CuSO₄ before preparation of extracts for assays. FIG. 17A: Northern blot analysis from RNA isolated from cultures used for the β-galactosidase assay in (FIG. 17B). Comparison of transcription and steady-state levels of the SEC61 and KAR2 mRNAs in vector-control, tunicamycin-treated and ΔCT-expressing strains: Northern blot analysis (FIG. 17C) and transcription run-on assays (FIG. 17D) of BY4733 cells transformed with pYEX-BX vector or pYEX-BX containing ΔCT. Transcription of KAR2 and SEC61 was induced in the vector-only strain by the addition of tunicamycin to 2 μg/ml (+Tm) for 2 hours prior to the run-on assay and harvesting of cells for preparation of RNA. Transcription and steady state levels of SEC61 and KAR2 mRNA were compared to those of the PYK1 gene. GFP was used as a control for non-specific binding in the run-on assay.

FIGS. 18A and 18B show that the half-life of SEC61 mRNA is increased in cells expressing p180. FIG. 18A: Northern blot of total cellular RNA extracted from BY4733 cells with a tetracycline-repressible promoter integrated at the SEC61 locus following doxycycline arrest of transcription. Strains were transformed with pYEX-BX (vector) or pYEX-BX containing ACT and grown to mid-log phase in the presence of 0.5 mM CuSO₄ prior to the addition of doxycyline to 5 μg/ml. Cells were harvested at the indicated times and RNA isolated for analysis. Levels of SEC61 mRNA were normalized to the PGK1 mRNA. Four independent time-course analyses were performed, and the resulting half-life represents an average of the four experiments. DOX: doxycycline. FIG. 18B: Same as in A, only examining SEC61 mRNA levels in the other p180 expressing strains (diagrammed in FIG. 14A). Values for half-lives, based on two separate experiments, are given in the text.

FIG. 19 shows that the half-life of GFP harboring the KAR2 signal sequence is increased in cells expressing p180. Northern blot of total cellular RNA extracted from BY4733 cells transformed with tetracycline-repressible constructs encoding GFP or a KAR2-signal-sequence GFP (KAR2ss-GFP). Decay rates were compared for strains harboring the pYEX-BX vector or pYEX-BX containing ACT. Strains were grown to mid-log phase in the presence of 0.5 mM CuSO₄ prior to the addition of doxycyline to 5 mg/ml. Levels of GFP mRNAs were normalized to the PGK1 mRNA. Four independent time-course analyses were performed, and the resulting half-life represents an average of the four experiments. DOX: doxycycline.

FIG. 20 shows that messenger RNAs that encode components of the secretory pathway are highly associated with membrane-bound polysomes in p180-expressing cells. SEY6210 cells transformed with the pYEX-BX (YEX) vector or YEX containing the ACT form of p180 were induced with 0.5 mM CuSO₄for 6 hours. Free and membrane-polysomes were isolated by differential centrifugation. RNA was isolated as described in materials and methods and probed for PGK1, PEP4, SEC61 and KAR2. The graphs for each gene represent relative mRNA levels where the mRNA from the free polysome fraction of YEX was set to 1. White bars: mRNA from free polysomes; Striped bars: mRNA from membrane-bound polysomes.

### DETAILED DESCRIPTION

This invention pertains to methods of increasing production/expression of a protein in a eukaryotic cell. In one aspect, this invention provides a process for increasing the intracellular transport or secretion of a desired protein in a eukaryotic host cell. In another aspect this invention provides a method of increasing the intracellular half-life of an mRNA which results in increased expression of a protein encoded by that mRNA.

In general it was discovered that upregulating the level of a ribosome receptor (e.g. p180) results in the proliferation of rough endoplasmic reticulum (RER) and consequently elevated secretion of proteins bearing a secretory signal sequence.

It was also a surprising discovery that upregulating the level of a ribosome receptor results in the increased intracellular half-life of mRNAs which, in turn, results in increased expression levels of proteins encoded by those mRNAs. Modification of an mRNA (that is normally translated in the cytosol) to enable its targeting to the ER (e.g., by incorporation of a secretory signal) caused an increase in its stability in the presence of a ribosome receptor. Membrane fractionation demonstrated that an increased stability of mRNAs targeted to the secretory pathway could be correlated with their increased association with the ER membrane in ribosome receptor-expressing cells.

In view of these discoveries, in certain embodiments, this invention provides methods of increasing the production and/or secretion of a protein. In one embodiment, this invention provides a method of increasing the secretion of a protein. This involves increasing the level of ribosome receptor which is accompanied by a proliferation of rough endoplasmic reticulum which is associated with increased protein secretion.

In another embodiment, this invention provides a method of increasing the half-life of an mRNA in the cell. This involves increasing the intracellular level of a ribosome receptor (e.g. p180). Increased mRNA half-life results in increased protein translation and thereby increased levels of the protein(s) encoded by those mRNA(s).

I. Increasing the Level of a Ribosome Receptor.

As indicated above, in various embodiments, this invention contemplates methods that involve increasing the level of a ribosome receptor in a cell. In preferred embodiments, the ribosome receptor is increased by either increasing the expression of an endogenous ribosome receptor and/or by expressing a heterologous ribosome receptor in the subject cell.

In certain embodiments, the level of the ribosome receptor is elevated by modifying the expression level of an endogenous ribosome receptor. This is readily accomplished by a number of means well know to those of skill in the art. For example, the native ribosome receptor promoter can be modified or substituted with, e.g. a constitutive promoter that upregulates expression of the ribosome receptor. Methods of modifying or replacing native promoters to alter expression of endogenous genes are well known to those of skill in the art (see, e.g., U.S. Pat. No. 5,272,071, and PCT Applications WO 91/09955, WO 93/09222, WO 96/29411, WO 95/31560, and WO 91/12650). Cell lines that express high levels of the endogenous ribosome receptor can also be produced by methods of directed evolution and/or systematic selection.

In addition, or as an alternative, the cell(s) of interest can transfected with a nucleic acid construct that encodes and expresses a ribosome receptor or an effective fragment, mutant, or isoform thereof. In various embodiments, the transfection can be stable or transient. In various embodiments, the transfected construct can integrate into the host cell genome (e.g., via homologous recombination) or can remain episomal.

A) Ribosome Receptor.

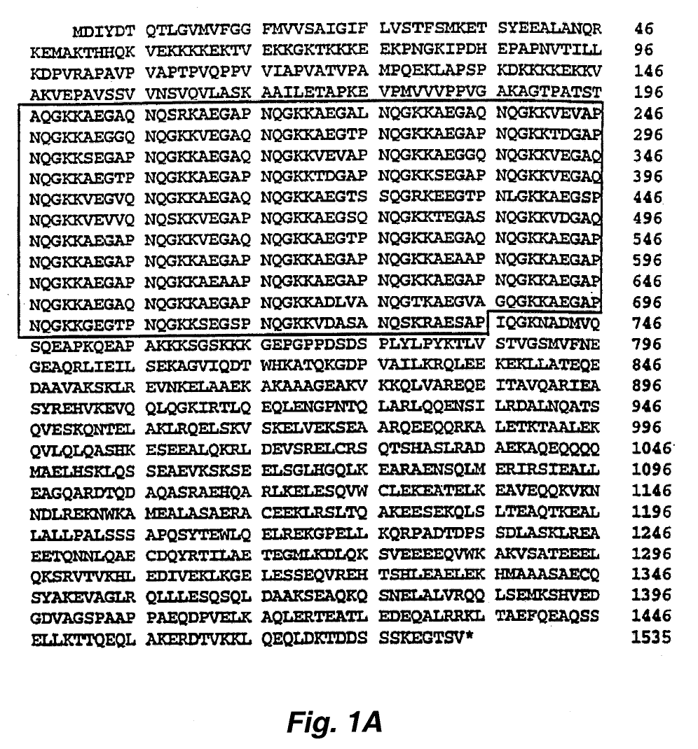

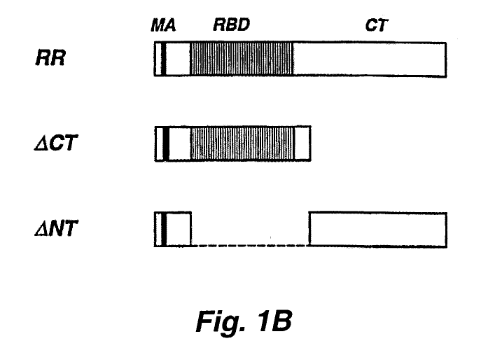

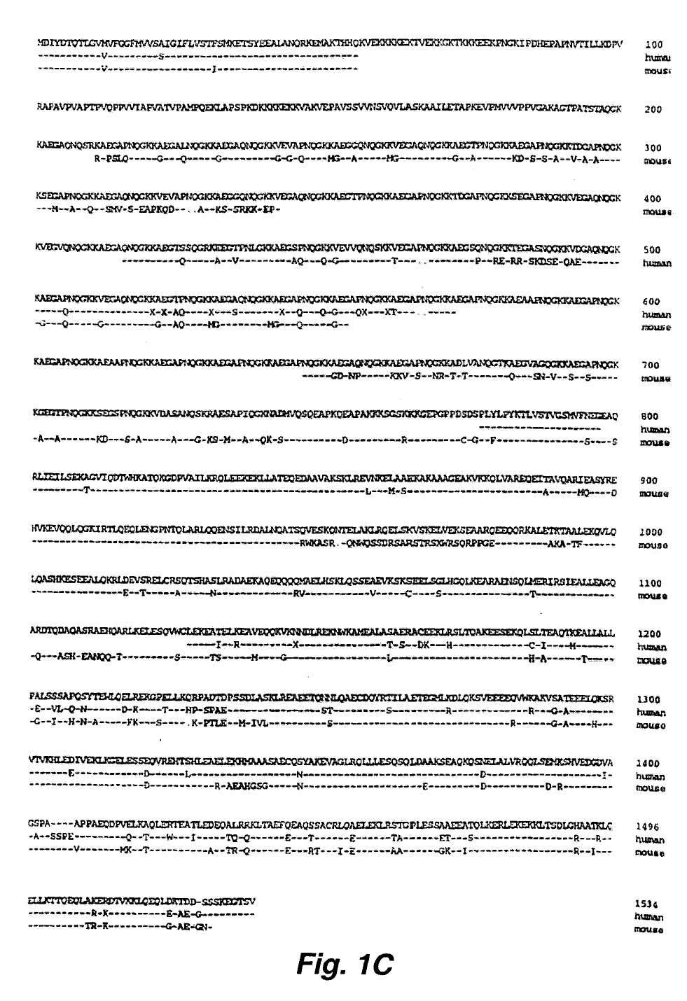

The ribosome receptor is well known and characterized for a wide variety of species. Thus, for example, the deduced primary structure of the 180 kD canine ribosome receptor (RRp) indicates three distinct domains: an amino-terminal stretch of 28 uncharged amino acids representing the membrane anchor, a basic region (pI=10.74) comprising the remainder of the amino-terminal halt and an acidic carboxyl-terminal half (pI=4.99). A striking feature of the amino acid sequence is a 10-amino acid consensus motif that is repeated 54 times in tandem without interruption in the amino-terminal positively charged region (Wanker et al. (1995) J. Cell Biol., 130: 29-39). The primary structure (amino acid sequence) of the RRp is shown in FIG. 1A. The nucleotide sequence for the RRP is available from GenBank as accession number X87224.

With reference to FIG. 1B, RR stands for full-length ribosome receptor, MA stands for the membrane-anchoring region, RBD stands for ribosome binding domain, CT stands for the carboxyl-terminal region. Shown as ACT is a truncation of the carboxyl-terminal domain, and shown as ANT is a construct harboring a deletion of the RBD. Further details, such as a restriction map, are described in Wanker et al. supra.

The primary structure of the ribosome receptor shown in FIG. 1A is deduced from the canine cDNA sequence. This protein appears very highly conserved in other species where sequence data is available. There is an ever-increasing number ribosome receptor sequences and fragments thereof appearing in the nucleic acid and protein databases. The most commonly posted mammalian sequences are from mouse and human sources. When translated into amino acids, a number of them align quite nicely with the sequence of the canine ribosome receptor. Due to its repetitive nature, it is difficult to align short ESTs within the N-terminal repeat-containing domain. However, as can be seen in FIG. 1C, a considerable number of human and mouse ESTs from non-repeat regions show a striking level of amino acid identity (>90%) over their entire length. For all of the ESTs shown in FIG. 1C, there are amino acid identities at 84% of the human and for 79% of the mouse sequences. At the very N-terminus of the protein, spanning the membrane-anchoring domain, amino acid identities between human, mouse, and canine sequences are in excess of 95%. The overall values for mouse are most likely even higher than 79% as the EST encoding amino acids 946 and 979 most likely has a sequencing error. Evidence from mouse genomic sequencing indicates that the mouse homologue has at least 45 of the 54 repeats found in the canine primary structure.

In view of the very highly conserved nature of the ribosome receptor as between species, and since we have demonstrated aspects of the invention in mammalian cells with a canine ribosome receptor fragment, the species from which the ribosome receptor or fragment originates in practicing this invention is not believed to be of great consequence.

As indicated above, a number of ribosome receptors other than the canine receptor have been isolated and/or sequenced. Such include, but are not limited to Mus musculus (GenBank Accession No: XM_{—}194049.1), Homo sapiens (GenBank Accession No: AL833822.1), and the like. Other species, mutant, truncations, and isoforms can readily be obtained from a GenBank similarity search, and/or by modification of known sequences.

Practice of the methods of this invention need not be limited to “native” or to full-length ribosome receptors. It is believed that truncated and/or mutated ribosome receptors and/or various ribosome receptor isoforms can prove as efficacious or even more efficacious in the methods of this invention than the wildtype ribosome receptor(s).

Effective fragments, mutants, isoforms, and the like can readily be identified by transfecting a cell with an appropriate nucleic acid construct that expresses the fragment/mutant/isoforms(s) of interest. Resulting proliferation of rough endoplasmic reticulum (RER) and/or increase in mRNA half-life can then be evaluated, e.g. as described in the examples herein.

B) Expression Systems.

In certain embodiments, the methods of this invention involve transfecting a cell (e.g. typically a eukaryotic cell) with a construct that encodes and expresses a ribosome receptor, and/or an effective fragment thereof and/or an effective mutant ribosome receptor, and/or an effective ribosome receptor isoforms in order to increase the intracellular level of the ribosome receptor, mutant, isoforms, or fragment. This will result in increased mRNA half-life in the cell, particularly of mRNAs encoding proteins targeted to a secretory pathway.

In certain embodiments, cells modified to express an increased level of ribosome receptor are used to express one or more endogenous proteins and/or are transfected with a nucleic acid construct that encodes a heterologous protein whose expression is desired. The heterologous protein can be tagged with a secretory signal sequence to induce the cell to secrete the desired protein product.

The methods of this invention are preferably carried out in essentially any eukaryotic cell-based expression system. Suitable eukaryotic cells include, but are not limited to fungi, plant, protozoan, invertebrate, or vertebrate cells. Expression of genes encoding desired proteins in genetically altered eukaryotic host cells can be accomplished by genetically engineered constructions and methods now well known to practitioners in this field.

Generally this involves creating a DNA sequence that encodes the desired protein (e.g. a ribosome receptor), placing the DNA in an expression cassette under the control of a particular promoter and transfecting that expression cassette into a eukaryotic host cell where the encoded protein(s) are expressed.

DNA encoding the ribosome proteins described herein can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Alternatively, the ribosome (or other polyepeptide) encoding sequences are cloned using appropriate restriction enzymes. The fragments may then be ligated to produce the desired DNA construct.

In certain embodiments, the nucleic acids of this invention can be cloned using DNA amplification methods such as polymerase chain reaction (PCR) (see, e.g., Example 1). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired (e.g. ribosome) sequence having terminal restriction sites. This nucleic acid can then be easily ligated into a vector containing a having the appropriate corresponding restriction sites. Suitable PCR primers can be determined by one of skill in the art using the sequence information provided herein. Appropriate restriction sites can also be added to the nucleic acid encoding the desired by site-directed mutagenesis. The plasmid containing the desired protein is cleaved with the appropriate restriction endonuclease and then ligated into an appropriate vector according to standard methods.

The nucleic acid sequences encoding ribosome receptors or other proteins can be expressed in a variety of host cells, preferably eukaryotic host cells including, but not limited to yeast, fungi, invertebrates (especially insect cells), and vertebrate, preferably mammalian such as the COS, CHO and HeLa cells lines and myeloma cell lines. The recombinant protein gene will be operably linked to appropriate expression control sequences for each host. For eukaryotic cells, the control sequences will typically include a promoter and often an enhancer (e.g., an enhancer derived from immunoglobulin genes, SV40, cytomegalovirus, etc.), and a polyadenylation sequence, and may include splice donor and acceptor sequences.

The plasmids (or other nucleic acid constructs) of the invention can be transferred into the chosen host cell by well-known methods such calcium phosphate treatment, electroporation, transfection reagents (e.g. lipofectin), or using viral vectors.

Widely used viral vector systems include, but are not limited to adenovirus, adeno associated virus, and various retroviral expression systems. The use of adenoviral vectors is well known to those of skill and is described in detail, e.g., in WO 96/25507. Suitable adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous genes into most mammalian cells.

Adeno-associated virus (AAV)-based vectors used to transduce cells with target nucleic acids, e.g., in the in vitro production of nucleic acids and peptides, and in in vivo and ex vivo gene therapy procedures are described, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

Cells transformed by the plasmids can be selected by resistance to antibiotics conferred by genes contained on the plasmids, such as the amp, gpt, neo and hyg genes.

The present invention also provides a eukaryotic-host cell expressing an elevated level of ribosome receptor. A wide variety of eukaryotic host cells are available for propagation and/or expression of vectors. Examples include, but are not limited to yeast or other fungal cells (including S. cerevesiae and P. pastoris), insect cells, plant cells, as well as higher eukaryotic cells (such as CHO cells and other mammalian cells).

In certain embodiments, the cells expressing elevated levels of ribosome receptor are maintained in culture. Typical culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

II. Expression of Endogenous or Heterologous Protein.

By practice of the methods of this invention, (e.g., increasing mRNA half-life), the capacity of capacity of eukaryotic cells (from yeast to human) to express and/or secrete particular endogenous and/or exogenous proteins can be dramatically increased. A ribosome receptor, or portions of the ribosome receptor, is expressed, e.g., in a stable manner in the desired cell type to produce an elevated level of ribosome receptor. These modified cells take on the attributes of highly differentiated secretory tissues such as pancreas, liver or plasma cells resulting in increased transport, processing, and secretion when compared to the unmanipulated original cell. In addition, these cells increase the half-life of transcribed mRNAs. These engineered cells then become the host for the high level production of correctly processed secretory and/or membrane-associated proteins of commercial importance. Expression levels of the ribosome receptor constructs can be adjusted to be optimal for the transport, processing and secretion of the protein of interest.

Typically, the modified host eukaryotic cells are also transfected with a gene or cDNA encoding the protein of interest whose expression and/or secretion is to be optimized/maximized. As will be understood, a number of categories of “desired proteins” exist, whose intracellular transport and/or secretion could be enhanced through the use of the invention. Most obvious are recombinant proteins whose expression is based on the transcription of cloned cDNA driven by a suitable promoter (e.g. a constitutive, inducible, or tissue-specific promoter). Such constructs are often, but not always, integrated into the genome of the host cell.

The cDNAs encoding the recombinant proteins are often derived from sources other than the host cell's complement of transcripts and would be defined as “heterologous” (e.g., a human cDNA expressed in CHO cells). Through the use of human host cells, homologous expression of human proteins could also be achieved. The transport, processing and secretion of artificial proteins, as exemplified by “humanized” monoclonal antibodies, can also be improved over existing technology through the use of the invention.

In practicing this invention, the expression of an increased amount of the desired protein may be inducible or non-inducible. Constitutive expression would typically be preferred in the routine commercial situation. However, as it is possible to optimize the synthesis of the protein whose expression is desired and vis a vis the synthesis of the machinery for transported, processing and secretion, by placing either the recombinant protein, and/or gene or cDNAs expressing the ribosome receptor under regulated control.

Once the desired protein is obtained in increased amounts with respect to wild-type eukaryotic host cell, then it preferably will be purified either from the growth medium of genetically engineered host cells or by extraction and purification from the host cells directly. In the case of secreted proteins, known technologies for isolation from growth media can be utilized. In the case of intracellular proteins, or membrane bound enzymes, many biochemical purifications have been worked out.

Further, in practicing the invention, the recombinant DNA molecule introduced into the genetic material of the eukaryotic host cell can be incorporated into the genome of the eukaryotic host cell or not. For the moment, the ability to create cell fines that achieve stable expression of proteins over long periods of time, a necessary prerequisite for FDA approval, is most readily achieved by integration into the host cell's chromosomes. Episomal (plasmid-based) transfection can lead to higher levels of expression, but can, in certain instances be unstable.

To direct the expressed polypeptide into the secretory pathway of a eukaryotic host cell, a secretory signal sequence (also known as a signal peptide, a leader sequence, prepro sequence or pre sequence) can be provided by the nucleic acid construct encoding the desired polypeptide. The secretory signal sequence is operably linked to a polypeptide-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

A wide variety of secretory signal sequences are known to those of skill in the art. These include, but are not limited to tissue-type plasminogen activator signal sequence (see, e.g., U.S. Pat. No. 5,641,655), secretory signals from IL-2 (MYRMQLLSCIALSLALVTNS (SEQ ID NO:1), see, e.g., Villinger et al. (1995) J. Immunol. 155:3946), a secretory signal from growth hormone (MATGSRTSLLLAFGLLCLPWLQEGSAFPT (SEQ ID NO:2), see, e.g., Roskam et al. (1979) Nucleic Acids Res. 7:30); a secretory signal from preproinsulin (MALWMRLLPLLALLALWGPDPAAAFVN (SEQ ID NO:3), see, e.g., Bell et al. (1980) Nature 284: 26); and influenza HA protein (MKAKLLVLLYAFVAGDQI (SEQ ID NO:4, see, e.g., Sekiwawa et al. (1990) PNAS 80: 3563), the signal leader sequence from the secreted cytokine IL-4, which comprises the first 24 amino acids of IL-4 as follows: MGLTSQLLPPLFFLLACAGNFVHG (SEQ ID NO:5), and the like.

Examples of suitable yeast signal sequences are those derived from yeast mating phermone αa-factor (encoded by the MFα1 gene), invertase (encoded by the SUC2 gene), or acid phosphatase (encoded by the PHO5 gene) (see, e.g., Romanos et al. (1995) Expression of Cloned Genes in Yeast, in DNA Cloning 2: A Practical Approach, 2nd Edition, Glover and Hames (eds.), pages 123-167 (Oxford University Press)).

III. Kits

In another embodiment this invention provides kits for increasing the half-life of an mRNA in a cell and/or kits for increasing the level of expressed and/or secreted protein(s). In certain embodiments, the kits comprise a container containing a nucleic acid construct encoding one or more ribosome receptors, and/or fragments thereof and/or mutants and/or isoforms thereof. In certain embodiments, the kits comprise a container containing a eukaryotic cell that has been treated or modified to express an elevated level of ribosome receptor as compared to an untreated or unmodified cell of the same type.

The kits optionally additionally comprise instructional materials teaching that increasing the intracellular level of a ribosome receptor will increase the intracellular half-life of an mRNA. In certain embodiments, the kits optionally comprise a vector for introducing a nucleic acid encoding the mRNA into the cell, and/or a transfection reagent for introducing the nucleic acid construct into said cell.

While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The invention will now be illustrated by the following examples and with reference to the Figures, which are intended to be illustrative and not limiting.

### EXAMPLES

The following examples are offered to illustrate, but not to limit the claimed invention.

### Example 1

### Preparation of Constructs and Expression of Proteins

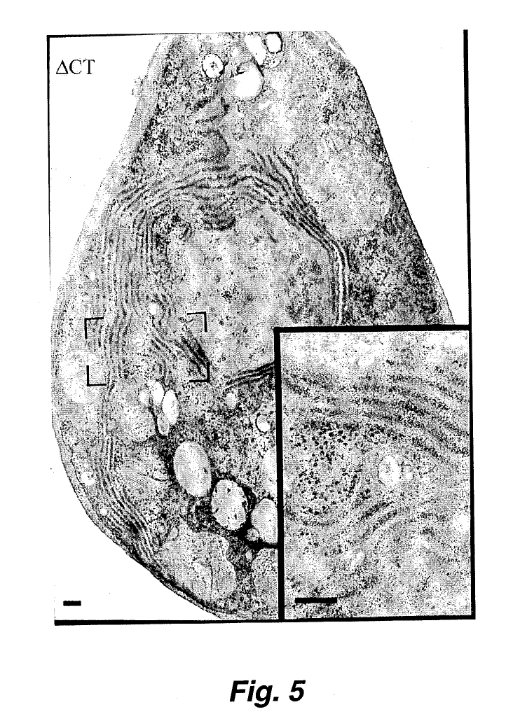

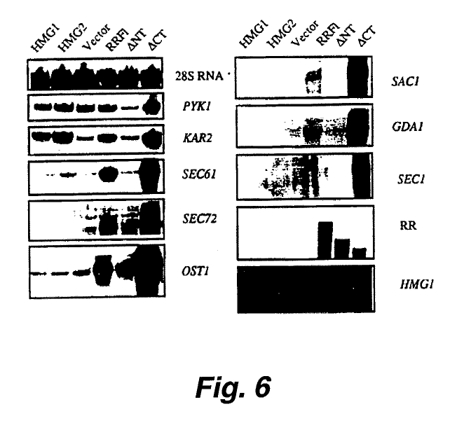

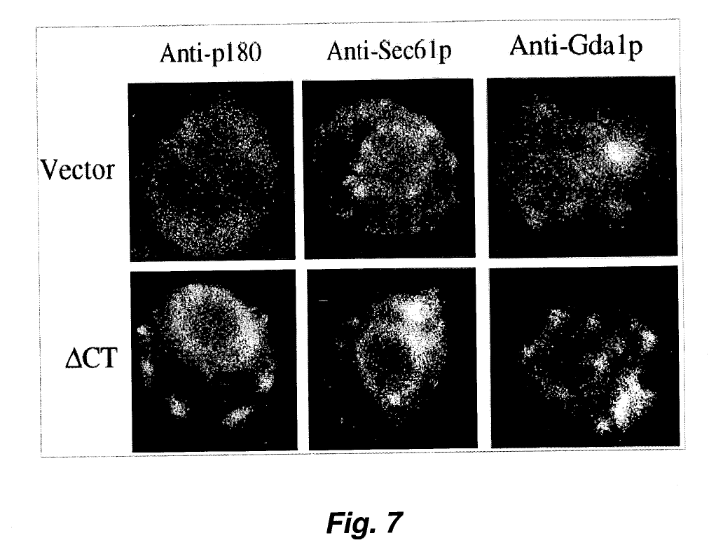

To achieve proliferation of rough ER membranes (as in FIG. 5) as well as other elements of the secretory pathway in yeast (FIGS. 6 and 7), the N-terminal half of the canine ribosome receptor (amino acids 1-826) was cloned into a pYEX-BX vector (Amrad Biotech) behind the promoter derived from the CUPI gene. The ribosome receptor was as described by Wanker et al. (1995) J. Cell Biol., 130: 29-39, in which a cDNA encoding the 180-kD canine ribosome receptor was reported to have been cloned and sequenced with the sequence data being available from GenBank/EMBL/DDBJ Accession No. X87224.

5×10⁷ cells were transfected with 5 μg of DNA using the published LiAc/DMSO procedure. Expression of the gene was then controlled by the presence or addition of copper (Cu₂SO₄, 0.5 mM) in the growth medium (SD).

Various other promoters can also be used in preparing constructs useful in practicing the invention. For example, instead of the CUP1 gene, ADH is another gene whose promoter could enable a high level of gene expression and represents a suitable equivalent. It may also be possible to induce equivalent, or even higher, levels of membrane proliferation by manipulating the constructs to contain only the ribosome binding (repeat-containing) domain and a membrane anchor. Alternatively, constructs with fewer or more repeats may enhance membrane production, and thus secretory activity.

### Example 2

### Co-Expression of a Heterologous Protein with a Ribosome Receptor

To demonstrate the invention in mammalian cells, full-length ribosome receptor cDNAs as well as the N-terminal half of the receptor (amino acids 1-826) were cloned into a pcDNA 3.1/Zeo vector (Invitrogen, San Diego, Calif.), where constitutive gene expression is controlled by the CMV promoter.

Transient transfections of mammalian cells (1.6×106 cells with 10 μg of DNA), were carried out through electroporation, prior to growth on DMEM (+10% FBS) in 10 cm dishes.

CHO cells were transfected by electroporation with ScaI linearized expression constructs to enable integration into the host cell genome. Cell lines stably expressing proteins cloned into the pcDNA 3.1 vector were selected through the inclusion of the antibiotic zeocin (250 μg/ml) in the growth medium. Other animal cells successfully responding to transfection by increasing their endoplasmic reticulums (ER) complement include HeLa (human cervical carcinoma), COS-7 (African green monkey kidney) and 293 (human embryonal kidney) cells.

Constitutive expression constructs: A ACT cDNA fragment was ligated into the pcDNA 3.1/Zeo (−) expression vector by standard techniques.

Inducible expression constructs: A ACT cDNA fragment was ligated into the ptND expression vector (Invitrogen) by standard techniques. In the resultant constructs, transcription was directed from the ecdysone/glucocorticoid response elements and the minimal heat shock promoter.

Secreted protein construct: Expression vectors encoding Secretory Alkaline Phosphatase (pSEAP2-Control) were obtained from Clonetech (Palo Alto, Calif.). These constructs were introduced into the appropriate cell type using lipofection techniques (Fugene6, Boehringer, Mannheim; Lipofectamine Plus, Life Technologies, Gaithersburg, Md.).

Cell culture conditions: CHO/K1 cells were grown in Ham's/F12 medium with 10% FBS. CHO-Ecr (ecdysone receptor-expressing) cells were grown in the same way as CHO/K1 but zeocin was added to a concentration of 250 μg/ml. 48 hours prior to transfection, cells were plated at 25% confluency, at 16 hours prior to transfection fresh medium was added, and in the case of CHO-Ecr, zeocin was omitted.

Transfections: 10 μg of linearized expression construct was introduced by electroporation into CHO/K1. or CHO-Ecr cells (Invitrogen). Cells (1.6×10⁶) were transfected in a final volume of 400 μl of culture medium. After the pulse, cells were transferred to 100 mm plates with 10 ml of culture medium. Following a 24-hour recovery time, medium was changed and selection applied by the addition of either 300 μg of zeocin (non-inducible constructs) or 250 μg/ml zeocin and 500 μg/ml G418 (inducible constructs). Cells were then allowed to grow for another 24 hours prior to clone selection.

Clone selection: Clone selection was carried out using high dilution and seeding in 96-well plates or by cylinder cloning discrete foci on 150 mm plates.

Cells were initially screened for overexpression of p180-related proteins by immunofluorescence using anti-p180 antibodies. After the initial screen, positive clones were expanded for further characterization.

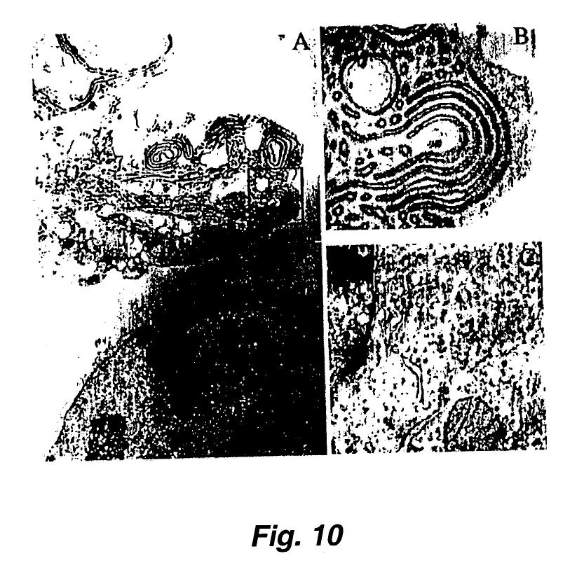

Assessment of transfection and upregulation of the secretory pathway was carried out morphologically as well as biochemically. The results of these studies are depicted in FIGS. 10-13 and described herein.

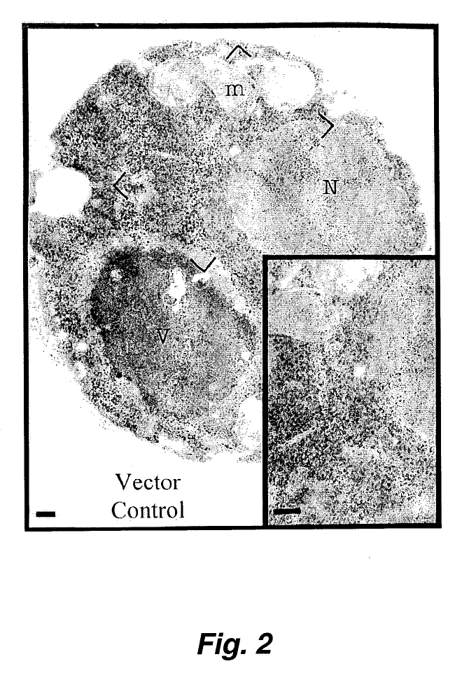

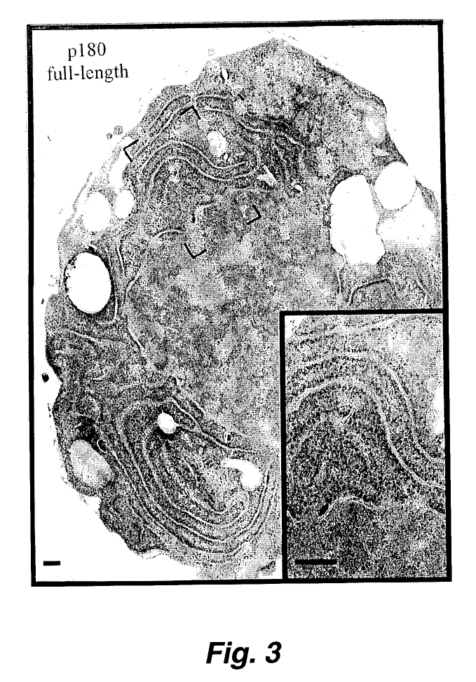

In wild type yeast cells, (FIG. 2) there is an overall lack of observable membranous structures in the cytoplasm. By comparing this electron micrograph to ones shown as FIGS. 3 and 5, dramatic changes are obvious in the cytoplasm.

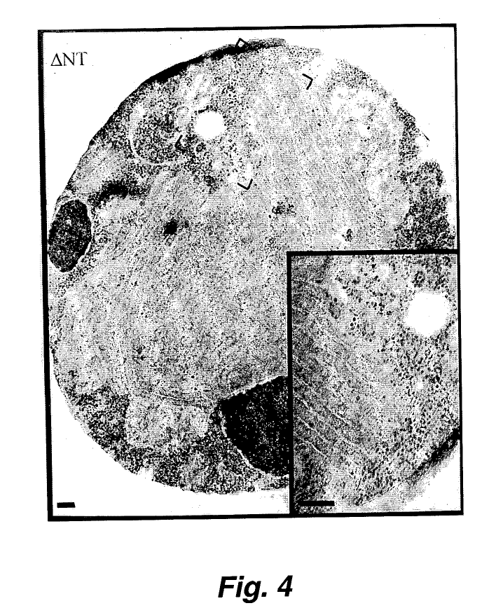

Large quantities of ribosome-studded membranes, reminiscent of rough endoplasmic reticulum and typical of actively secreting tissues, have appeared. In FIG. 4, where the ribosome binding domain has been deleted from the receptor that is being expressed, large quantities of membranes are still produced, as in FIG. 3, however there are no densely-stained particles (ribosomes) associated with the membranes. In FIG. 5, where yeast have been transfected with a construct encoding little more than the repeat domain and the membrane anchor, the greatest proliferation of rough membranes has occurred, leaving little if any unoccupied space in the cytoplasm.

It appears that the membranes induced and seen clearly in FIGS. 3 and 5 represent rough ER. Further evidence supporting the notion that these membranes represent bona fide rough ER is shown in FIG. 6. The expression of constructs that induce rough membranes induces the co-expression of genes encoding rough ER proteins. Moreover, the expression of genes encoding marker proteins further along the secretory pathway occurs in the case of membrane induction by the ACT construct. The uniqueness of the ability of ACT to selectively induce these genes is demonstrated by the relatively low ability of other constructs—some of which induce smooth membrane proliferation in yeast (e.g., ANT)—to enable similar changes.

FIG. 7 documents the fact that the proteins whose genes are expressed at higher levels in induced cells (FIG. 6) are also synthesized and incorporated into the correct organelle is demonstrated in FIG. 7 direct immunofluorescence using the appropriate antibodies show an upregulation in the appearance of ER, complete with one of its resident proteins (Sec61p), as well as an increase in the number of Golgi-like structures in the transfected cells containing the resident guanosine diphosphatase (Gda1) protein. Taken together, the data presented in FIGS. 6 and 7 indicate that genes of the secretory pathway are expressed and incorporated correctly into organelles during membrane proliferation induced by the expression of (part of) the ribosome receptor.

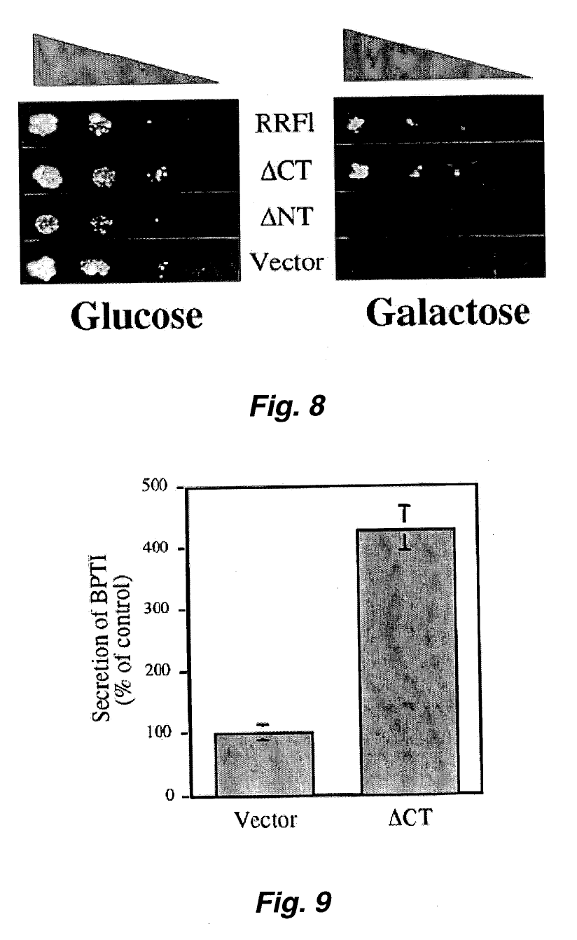

FIG. 8 is a growth assay demonstrating that cells expressing high levels of a gene encoding the secretory protein bovine pancreatic trypsin inhibitor (BPTI) inducible GAL promoter, growth stopped when BPTI production was induced in wild-type cells (vector only) (FIG. 8). It is likely that this block was the result of blocking the secretory pathway with excess BPTI. The block was relieved in the case of cells where membrane proliferation (and hence the secretory pathway) was induced through expression of RED containing constructs (full-length RRp and ACT). Membrane proliferation alone, as occurs in the case of the expression of ANT, was incapable of rescuing these cells from the BPTI block.

That the BPTI block was relieved by its secretion from the cells that had proliferated the secretory pathway is demonstrated in FIG. 9. In this case, a sensitive assay for BPTI's ability to inhibit the proteolytic cleavage of a substrate by trypsin indicated that a 400% increase in BPTI secretion had taken place in ACT-containing cells, compared to vector-only controls. This value may represent a minimum value in the cell's ability to upregulate its secretory capacity as neither the levels of BPTI, nor of ACT have been optimized as yet.

That which was observed in yeast can also occur in mammalian cells. The proliferation of rough membranes in the cytoplasm of transfected African monkey kidney (COS-7) cells is dramatically demonstrated in the electron micrographs displayed in FIG. 10. Panel A shows two cells at low magnification, one transfected (top) and one untransfected (bottom). Higher magnification of the transfected cell (panel B) shows extensive rough ER proliferation compared to the untransfected cell (panel C). In this case, the full-length ribosome receptor was used, however immunofluorescence data show that similar membrane proliferation occurs using the ACT construct (not shown). Double label immunofluorescence studies verified that expression of the ribosome receptor in these cells results in the proliferation of rough ER membranes that contain increased levels of other resident proteins whose synthesis appears induced in response to the transfection (not shown).

As many commercial applications rely on the use of Chinese hamster ovary cells (CHO) for secretion of important proteins, we established two CHO cell lines that were stably transfected with constructs encoding ACT as the inducer of membrane proliferation. In the first case, a vector was used that places ACT under control of the constitutive human cytomegalovirus (CMV) promoter, whereas the second construct placed ACT behind an inducible hybrid promoter that is stimulated through including the insect hormone ecdysone or ecdysone-analogs in the growth medium.

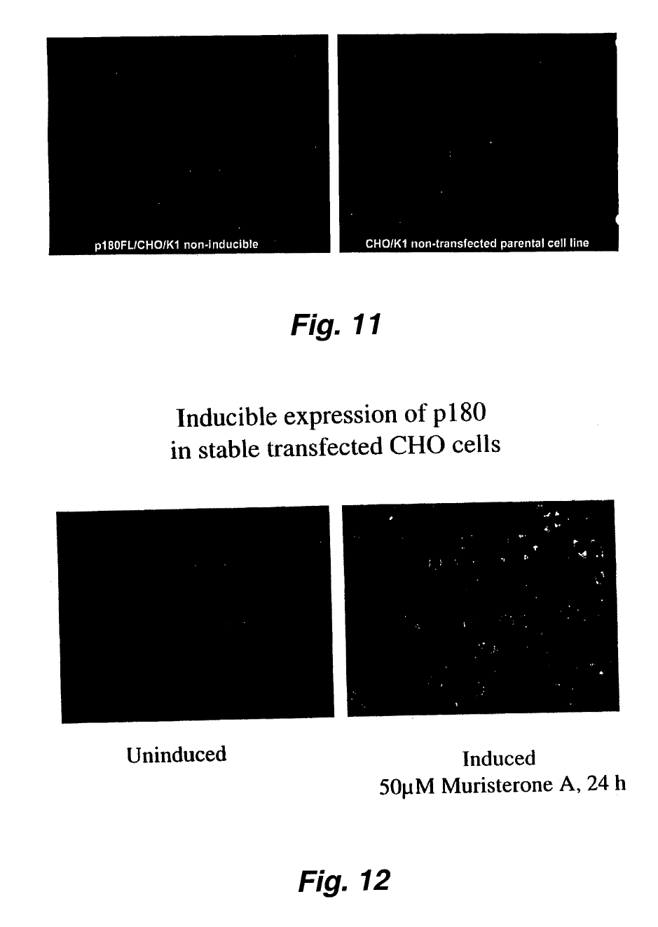

FIG. 11 shows immunofluorescent detection of the ribosome receptor in Chinese hamster ovary (CHO) cells that have been stably transfected with cDNA encoding the ribosome receptor. Note extensive membrane proliferation in transfected cells. Left panel: Cells transfected with cDNA encoding the ribosome receptor. Right panel: Cells transfected with vector alone.

FIG. 12 shows immunofluorescent detection of the ribosome receptor in Chinese hamster ovary (CHO) cells that have been stably transfected with a cDNA construct that enables inducible expression of the ribosome receptor. Left panel: Cells that have not been induced to express the ribosome receptor. Right panel: Cells grown for 24 hours in the presence of inducer.

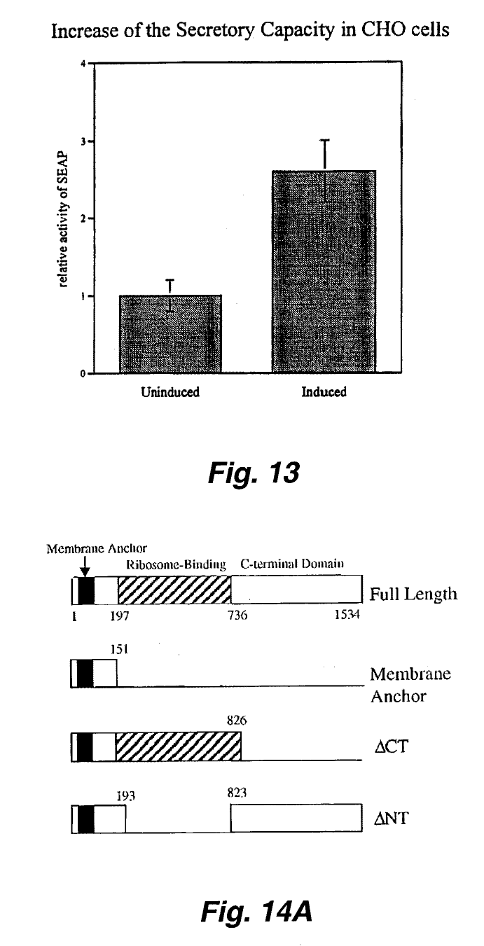

FIG. 13 illustrates results of an assay for secreted alkaline phosphatase where cells described in FIG. 12 were transiently transfected with a plasmid encoding a secreted form of the enzyme alkaline phosphatase. Quantities of the enzyme appearing the culture medium at 24 hours after induction were measured colorimetrically for control cells and for ones whose expression of the ribosome receptor had been induced.

FIGS. 11 and 12 document the proliferation of the endoplasmic reticulum in cells that express ACT. In the case of cells whose membrane proliferation was driven by constitutive expression of ACT driven by the CMV promoter, an abundant rough ER staining pattern is observed in comparison to vector-only controls (FIG. 11). A similar picture was obtained in the case of cells stably transfected with ACT under control of the inducible promoter. In this instance, striking upregulation of rough ER was observed after 24 hours of growth in the presence of the inducer when compared to uninduced control cells (FIG. 12).

As was the case for yeast, we demonstrated that mammalian cells manipulated in the manner of the invention have an increased ability to secrete recombinant proteins. Thus, a secretory form of the enzyme alkaline phosphatase, under the control of the constitutive SV-40 promoter, was transfected into the inducible CHO cell line described above. Experiments summarized in FIG. 13 indicate a two- to three-fold increase in secretion in cells upon induction of the gene encoding ACT. This is believed to be a minimum value for the increase in secretion, with greater levels achievable when the levels of alkaline phosphatase and the levels of ACT expression have been optimized.

### Example 3

### Induction of Secretory Pathway Components in Yeast is Associated with Increased Stability of their mRNA

Abstract

The over-expression of certain membrane proteins is accompanied by a striking proliferation of intracellular membranes. One of the best characterized inducers of membrane proliferation is the 180 kDa mammalian ribosome receptor (p180), whose expression in yeast results in increases in levels of mRNAs encoding proteins that function in the secretory pathway, and an elevation in the cell's ability to secrete proteins. In this study we demonstrate that neither the unfolded protein response (UPR) nor increased transcription accounts for membrane proliferation or the observed increase in secretory pathway mRNAs. Rather, p180-induced up-regulation of certain secretory pathway transcripts is due to a p180-mediated increase in the longevity of these mRNA species, as determined by measurements of transcriptional activity and specific mRNA turnover. Moreover, we show that the longevity of mRNA in general is substantially promoted through the process of its targeting to the membrane of the endoplasmic reticulum. With respect to the terminal differentiation of secretory tissues, results from this model system provide insights into how the expression of a single protein, p180, could result in substantial morphological and functional changes.

Introduction.

A number of internal and external stimuli trigger the expansion of the endoplasmic retiuculum (ER) membrane system and stimulate increases in levels of ER-localized proteins in both mammalian cells and in the yeast S. cerevisiae. Particularly dramatic is the proliferation of membranes that occurs during the terminal differentiation of secretory cells. In the case of mammalian tissues with extremely high secretory capacity, such as pancreas or liver, the concentration of an integral ER ribosome receptor (p180) is especially high. Expression of p180 in yeast, a cell without a structural p180 homologue, leads to a proliferation of rough ER membranes accompanied by an increased secretory capacity (Becker et al. (1999) J Cell Biol 146: 273-284; Wanker et al. (1995) J Cell Biol 130: 29-39.). Overexpression of numerous proteins, including endogenous yeast membrane proteins such as the HMG CoA reductase isoforms (Hmg1p and Hmg2p) or the peroxisomal protein Pex15p, results in the proliferation of smooth membranes (Koning and Roberts (1996) Mol Biol Cell 7: 769-789; Parrish et al. (1995) Mol Biol Cell 6: 1535-1547; Wright et al. (1988) J Cell Biol 107: 101-114; Elgersma et al. (1997) Embo J 16: 7326-7341). Membrane proliferation and elevated levels of ER-localized chaperones is also associated with expression of certain forms of cytochrome P450 (Menzel et al. (1996) Arch Biochem Biophys 330: 97-109; Menzel et al. (1997) Yeast 13: 1211-1229). Only in the case of p180 expression, however, have increases in rough membranes as well as secretory capacity been reported (Becker et al. (1999) J Cell Biol 146: 273-284).

The proliferation of certain ER-specific proteins results through disturbances within the lumen of the ER sensed via a well-characterized mechanism known as the unfolded protein response (UPR). The UPR represents a signal transduction pathway in yeast that links stress in the ER to transcription of genes encoding ER-resident proteins (Patil and Walter (2001) Curr Opin Cell Biol 13: 349-355; Spear and Ng (2001) Traffic 2: 515-523). Several similarities exist between induction of the UPR and proliferation of ER membranes upon expression of p180. In both processes, the levels of mRNAs that encode ER-localized chaperones and phospholipid biosynthetic genes are elevated (Becker et al. (1999) J Cell Biol 146: 273-284; Cox et al. (1997) Mol Biol Cell 8: 1805-1814). Furthermore, p180-induced membrane proliferation is accompanied by increased levels of secretory pathway mRNAs, another feature of cells undergoing a UPR (Becker et al. (1999) J Cell Biol 146: 273-284; Travers et al. (2000) Cell 101: 249-258). Increased secretory capacity, a prominent feature of p180 expression, has not been reported as a consequence of the UPR.

Cells that are deleted for IRE1 are unable to undergo a UPR. Through analysis of such mutants, the UPR has been linked to membrane-related cellular processes such as phospholipid synthesis, protein modification and secretion, and ER-associated protein degradation (Cox et al. (1997) Mol Biol Cell 8: 1805-1814; Travers et al. (2000) Cell 101: 249-258; Friedlander et al. (2000) Nat Cell Biol 2: 379-384). These mutational analyses, coupled with the observation that levels of ER-localized proteins were elevated in the UPR, led us to investigate the relevance of the UPR to membrane proliferation stimulated by p180-expression in yeast. We conclusively demonstrate in this study that p180-induced membrane proliferation as well as increases mRNA levels of ER-localized proteins and phospholipid biosynthetic enzymes occurs by an Ire1p-independent mechanism.

Instead, we report here the involvement of a novel mechanism that potentially regulates the increased abundance of secretory pathway component transcripts observed during p180-induced membrane proliferation. Our results show that p180 expression stabilizes secretory component mRNAs, and that targeting of any mRNA to the ER membrane is capable of mediating a substantial increase in its longevity. The conclusions we draw from our work in this model system could provide significant insights into processes occurring during the terminal differentiation of mammalian secretory tissues.

Results

P180-Induced Membrane Biogenesis and Elevated Levels of Secretory and Lipid Biosynthetic mRNAs Occurs Independently of the Unfolded Protein Response.

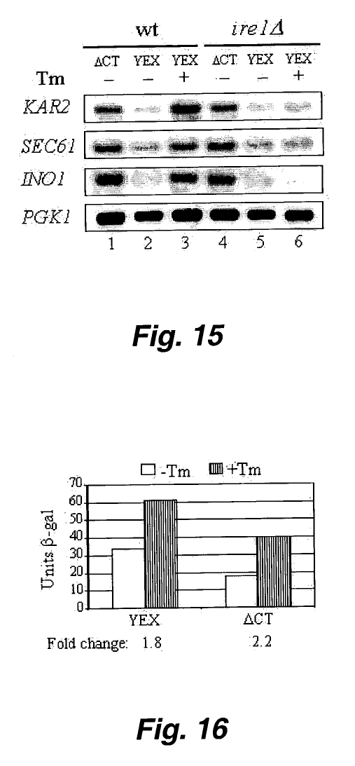

To determine if IRE1 is required for p180-induced membrane proliferation, a strain was created in which signaling through the UPR was disabled by replacing the entire coding region of IRE1 with the HIS3 gene. This Δire1 strain was found to be hypersensitive to agents that lead to the accumulation of unfolded proteins in the ER, such as treatment with the glycosylation inhibitor tunicamycin (data not shown). As shown previously (Cox et al. (1997) Mol Biol Cell 8: 1805-1814), these Δire1 cells were unable to induce transcription of genes such as KAR2 and INO1 that are known to increase during treatment with tunicamycin (FIG. 15, compare lanes 3 and 6).

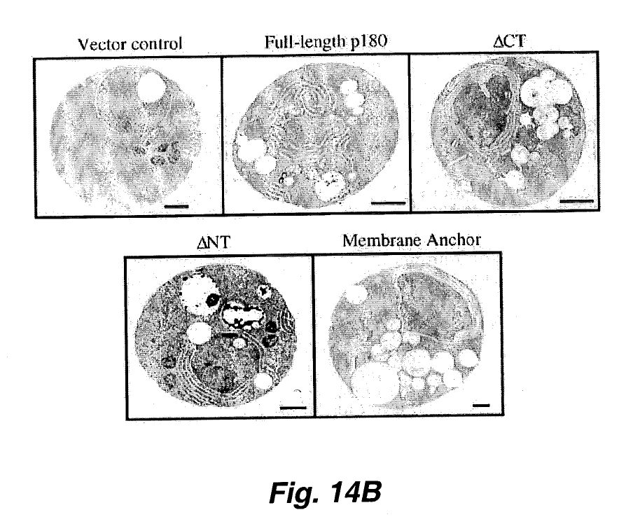

We first asked if the hallmark of p180 expression, proliferation of ER membranes, is affected in an Δire1 strain. Various p180 constructs (FIG. 14A) were expressed in the Δire1 strain from the high copy pYEX-BX plasmid under the control of the strong, inducible CUP1 promoter. Proliferation of intracellular membranes was examined by electron microscopy. The micrographs indicate that the status of the IRE1 gene had no bearing on membrane proliferation in p180-expressing cells, as expression of the various p180 constructs in Δire1 cells induced membranes with the same morphologies as in wild type cells (FIG. 14B). All constructs induced some form of membrane, either perinuclear or membranes located in the periphery of cells, or both. Full-length p180 and ANT induced membranes with the characteristic 80-100 nm spacing (Becker et al. (1999) J Cell Biol 146: 273-284), while the ACT and MA constructs, which lack the COOH-terminal domain, induced membranes with much closer spacing. These results suggest p180-induced membrane proliferation may occur in a UPR-independent fashion.

In all cases where it was examined, IRE1 was required for the upregulation of chaperones such as Kar2p. We were therefore eager to ascertain the requirement of UPR signaling in the p180-induced upregulation of mRNAs encoding secretory pathway and phospholipid biosynthetic genes, including KAR2. The steady state levels of these mRNAs in the Δire1 strain were examined. Of the p180 constructs previously characterized (Becker et al. (1999) J Cell Biol 146: 273-284; Wanker et al. (1995) J Cell Biol 130: 29-39), the ΔCT construct, comprised of the membrane-spanning and ribosome-binding regions (FIG. 15A), produced the largest increase in these mRNAs. Accordingly, ΔCT was used to study the relationship between this aspect of the p180 phenotype and the UPR. RNA was isolated for Northern blot analysis from wild type and Δire1 cells harboring the empty pYEX-BX vector in the presence or absence of tunicamycin and yeast expressing the ΔCT form of p180. Genes involved in translocation (SEC61), protein folding in the ER (KAR2) and inositol phospholipid biosynthesis (INO1) were compared for each strain. A housekeeping gene, phosphoglycerate kinase (PGK1), was used as a control. Expression of ΔCT in both wild type and Δire1 strains induced comparable steady state mRNA levels of KAR2, SEC61, and INO1 (FIG. 15: compare lanes 1 and 2 with lanes 4 and 5). Deletion of IRE1, however, abolished upregulation of these mRNAs in response to tunicamycin. These results are consistent with the supposition that the cellular response to p180 induction is very likely UPR-independent.

Genes whose transcription is induced by the UPR contain a 22-base pair UPR responsive element (UPRE) in their upstream regulatory regions (Kohno et al. (1993) Mol Cell Biol 13: 877-890; Mori et al. (1992) Embo J 11: 2583-9253). To confirm that the expression of p180 does not induce the UPR, assays measuring activity at this 22-base pair UPRE were performed. The plasmid pMCZ-Y, which encodes a fusion between the UPRE of the KAR2 gene, minimal CYC1 promoter elements and the coding sequence for the E. coli β-galactosidase enzyme, has been used previously for such assays (Mori et al. (1996) Genes Cells 1: 803-817). As expected in cells harboring pMCZ-Y, increased β-galactosidase activity was seen as a result of tunicamycin treatment. In extracts from cells transformed with the pYEX-BX vector and pMCZ-Y,β-galactosidase activity increased approximately 2-fold upon 2-hour treatment with 2 μg/ml tunicamycin (FIG. 16). In contrast, expression of ΔCT in pMCZ-Y-harboring cells slightly lowered β-galactosidase activity. ΔCT-expressing cells did not show a compromised UPR, as β-galactosidase activity was increased approximately 2-fold in extracts from tunicamycin-treated ΔCT cells compared to untreated ΔCT cells. Taken together, these data provide conclusive evidence that expression of ΔCT does not induce the unfolded protein response, nor is the UPR required for the p180-associated increases in levels of secretory pathway mRNA or intracellular membrane proliferation.

Transcriptional Regulation is Not Responsible for Increased Levels of Certain Secretory Pathway mRNAs in p180-Expressing Cells.

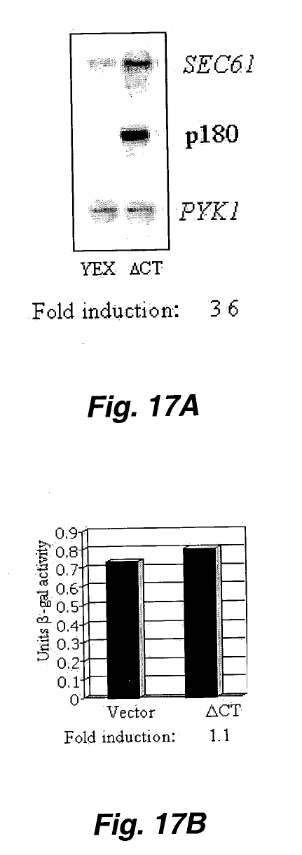

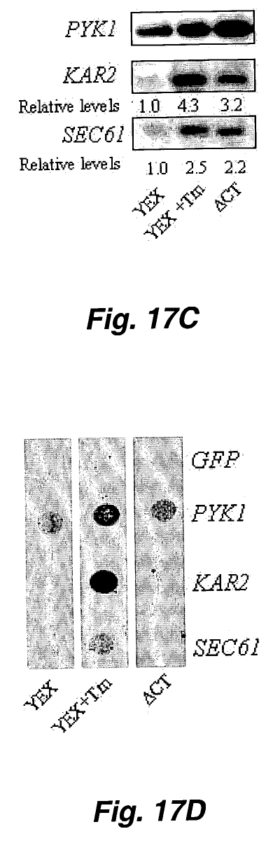

An alternative mechanism governing the p180-induced increase in levels of mRNAs encoding secretory pathway proteins might be the activation of a second signal transduction pathway, analogous to the UPR. Such a pathway would link the endoplasmic reticulum and the nucleus and result in increased transcription of genes encoding secretory pathway proteins. To determine if such a pathway exists, fusions were created between the promoter of a p180-induced gene (SEC61) and the E. coli LacZ gene. Levels of the SEC61 mRNA show significant increases due to the expression of p180 (FIG. 17A), and if these increases are due to transcriptional induction, increased activity at the SEC61 promoter should be measurable by increases in activity of the β-galactosidase enzyme. Surprisingly, no significant difference was observed in β-galactosidase activity between vector control cells and those expressing ΔCT (FIG. 17B). (Although unlikely, we cannot formally exclude the possibility that the SEC61-LacZ construct does not contain crucial elements SEC61 promoter, should these lie outside the 1000 base-pair segment 5′ to the SEC61 coding region that we fused to LacZ,). A testable alternative hypothesis is that the changes in the level of the SEC61 mRNA, and presumably other secretory pathway mRNAs in p180-expressing cells, occur not because of increased transcription; rather, the changes reflect decreases in mRNA degradation.

To assess the actual transcription levels of the SEC61 and KAR2 genes, we used a transcription run-on assay. Transcription of these genes was normalized against transcription of the pyruvate kinase (PYK1) gene, whose mRNA levels remain unchanged in the presence of p180. The gene encoding green fluorescent protein (GFP), which is not present in yeast, was used as a control for non-specific binding of radiolabeled transcripts. Northern analysis showed that expression of ACT resulted in an increase in the steady state levels of the SEC61 and KAR2 mRNAs similar to those caused by induction of the UPR (YEX+Tm cells) (FIG. 17C). However, increased transcription of these genes was not observed in the run-on assay (FIG. 17D), in contrast to tunicamycin-treated cells. These data show that increases in the steady-state levels of KAR2 and SEC61 mRNA caused by p180 expression are not due to transcriptional activity. We propose that a decrease in mRNA turnover is responsible.

P180 Stabilizes mRNAs that are Targeted to the Secretory Pathway

To directly measure the turnover of the SEC61 mRNA, transcription of the endogenous SEC61 gene was placed under the control of a tetracycline-repressible promoter, creating a means to halt transcription and monitor decay of this mRNA species. The entire DNA sequence between the start codon of the upstream divergently transcribed CSR1 gene and the SEC61 start codon was replaced with elements allowing control of SEC61 expression by tetracycline. This tet-SEC61 strain produced approximately 90% of wild-type levels of SEC61 mRNA in the absence of added drug (data not shown). Upon addition of the tetracycline analogue, doxycyline, transcription of the SEC61 gene ceases. Decay of the SEC61 mRNA was monitored by quantitative Northern blotting in a time-course following drug addition in cells expressing the ΔCT construct and vector-control cells. PGK1 mRNA is a housekeeping gene whose expression is not affected by p180 and was thus used as a control (LaGrandeur and Parker (1999) Rna 5: 420-433).

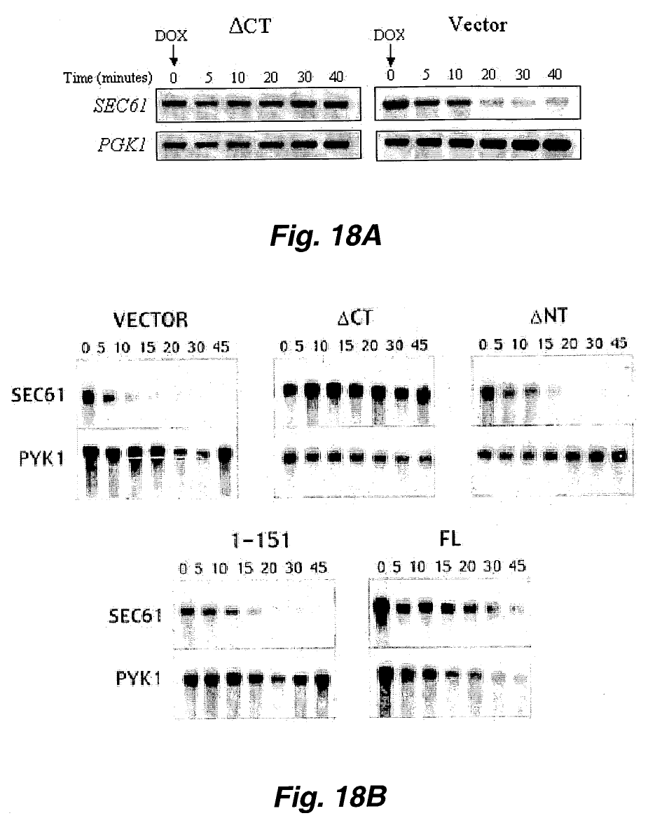

In vector-control cells, the half-life of SEC61 mRNA was approximately 5 minutes, whereas in ACT-expressing cells, nearly all of the SEC61 mRNA remained after 40 minutes (FIG. 18). This value may be conservative, as one of our experiments indicated the half-life for SEC61 mRNA in ACT-expressing cells was in excess of 100 minutes (not shown). These data demonstrate a profound increase in the stability of SEC61 mRNA in p180-expressing yeast.

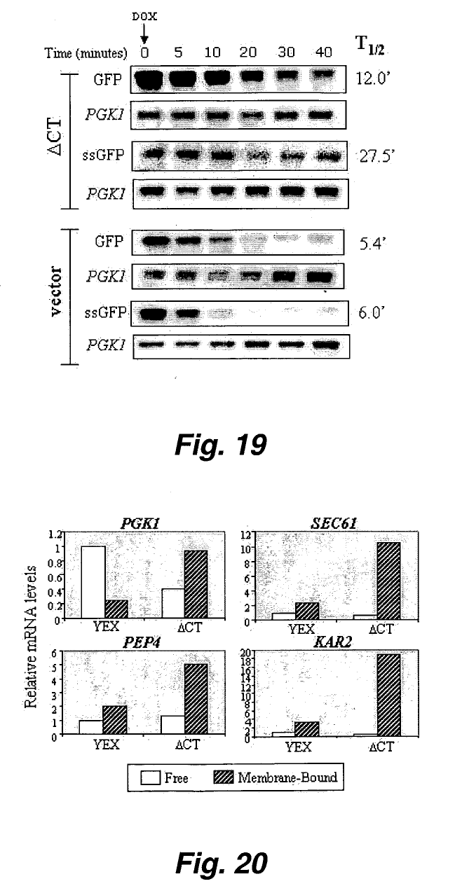

Since a number of secretory pathway mRNAs (which are translated on the endoplasmic reticulum) are upregulated by expression of p180 (Becker et al. (1999) J Cell Biol 146: 273-284), one hypothesis is that targeting these mRNAs to the endoplasmic reticulum is sufficient to cause a decrease their turnover in a manner dependent on the ER-localized p180. The turnover rate of constructs that differ only in the presence of an ER-targeting signal could be used to determine if ER-targeting is sufficient to reduce the rate of decay of an mRNA that is normally localized to the cytoplasm. Two constructs were generated: one that encodes GFP, and another that encodes a fusion of the KAR2 signal sequence (KAR2ss) to GFP, yielding KAR2ss-GFP. These constructs were generated in pCM182 (Gari et al. (1997) Yeast 13: 837-848), such that transcription of these constructs is under the control of a tetracycline repressible promoter. Rates of decay of the GFP and KAR2ss-GFP mRNA were compared in vector-control and ΔCT-expressing cells. KAR2ss-GFP mRNA had a half-life of 6 minutes in the vector control strains, and a half-life of 27.5 minutes in ACT-expressing cells, indicating about 4.5-fold decrease in the decay rate of the mRNA in ACT-expressing cells (FIG. 19). The GFP mRNA, had a half-life of 5.4 minutes and 12 minutes in vector-control and ACT-expressing cells, respectively, only slightly more than a 2-fold difference in half-life for non-targeted GFP mRNA in ΔCT-expressing cells. These results suggest that ΔCT preferentially stabilizes ER-targeted mRNA species.

The fact that the half-life of the ER-targeted GFP mRNA increased substantially led us to investigate whether mRNAs exhibiting increased longevity are indeed physically associated with the site of p180 localization, the rough ER. Accordingly, we undertook an analysis of mRNA distribution on membrane-bound and free polysomes derived from vector control and ΔCT-expressing cells. RNA was isolated from free polysomes (FP) and membrane-bound polysomes (MBP) by taking advantage of the different sedimentation properties of membrane and soluble fractions (see Materials and Methods). Probes were generated from fragments of KAR2 and SEC61, as well as from the coding regions of PEP4 and PGK1. PEP4, which encodes a vacuolar protease, is targeted to the secretory pathway and is used as a marker for MBP-specific mRNA. PGK1 mRNA encodes the soluble phosphoglycerate kinase that is present primarily in FP-containing fractions. The histograms in FIG. 20, derived from quantitative Northern blotting, demonstrate that the increased amounts of KAR2 and SEC61 mRNAs in ΔCT-expressing cells are almost exclusively associated with their presence in polysomes derived from the membrane fraction. As expected, PEP4 mRNA was also determined to be primarily associated with the MBP fraction, and increased slightly upon ΔCT-expression. Interestingly, the p180-unresponsive PGK1 mRNA that was present primarily in the FP fraction of vector-expressing cells (as expected), showed a slight shift in its distribution towards the MBP fraction in ΔCT-expressing cells. This may be a consequence of the overall increase in membrane-bound ribosomes in ΔCT-expressing cells that we observed previously.

This yeast model reproduces many of the events associated with the acquisition of a high secretory capacity, and may reflect a conserved mechanism that occurs during cellular differentiation in other organisms. It will thus be interesting to determine whether p180 plays a similar role in the terminal differentiation of secretory cells in higher eukaryotes.

Discussion

The work presented here describes a novel, and UPR-independent, means for increasing ER components involving featuring a p180-mediated stabilization of mRNAs on the ER membrane. This finding sets the stage for future studies into the actual molecular dynamics of such a process. Deletion of IRE1, which encodes an essential signaling molecule of the UPR, hindered neither the proliferation of p180-inducible membranes nor the upregulation of mRNAs encoding various secretory pathway and phospholipid biosynthetic markers. We demonstrated that the increased level of SEC61 mRNA in p180-expressing cells was not regulated at the transcriptional level, but rather at the level of mRNA turnover. Modification of an mRNA (that is normally translated in the cytosol) to enable its targeting to the ER caused an increase in its stability in the presence of p180. Membrane fractionation demonstrated that an increased stability of mRNAs targeted to the secretory pathway could be correlated with their increased association with the ER membrane in p180-expressing cells.

In contrast to the induction of ER chaperones such as KAR2 in response to ER stress, the requirement for IRE1 for membrane proliferation is a subject of some debate. One group observed that IRE1 was essential for KAR2 mRNA induction in response to cytochrome p450 overexpression; however, it was not necessary for the production of ER-like membranes that arise upon expression of the protein (Menzel et al. (1997) Yeast 13: 1211-1229). A subsequent report demonstrated that IRE1 was essential for the proliferation of cytochrome p450-induced membranes, and revealed that differences in strain background accounted for the conflicting results (Takewaka et al. (1999) J Biochem (Tokyo) 125: 507-514). In our experiments, the IRE1 gene was deleted in two non-isogenic strains and found to be dispensable for p180-induced membrane proliferation and elevated levels of KAR2 (L. Block-Alper, M. Hyde; unpublished observations).

Our results also address the role of Ire1p in the inositol response (IR) and in membrane biogenesis. Phosphatidylinositol is one of the major lipids of membranes in eukaryotic cells. An understanding of any type of membrane proliferation will depend upon an elucidation of how the need for additional membrane lipid is met. There appear to be multiple mechanisms by which a key element in this lipid's metabolism, the INO1 gene, is regulated. It has been shown that deletion of IRE1 results in inositol auxotrophy, suggesting that the UPR and phospholipid biosynthesis may be linked (Nikawa and Yamashita (1992) Mol Microbiol 6: 1441-1446). Moreover, wild type cells that were induced to undergo a UPR had increased INO1 transcription, and deletion of OPI1, a gene that encodes an inhibitor of inositol phospholipid synthesis restored inositol prototropy to the Δire1 strain (Cox et al. (1997) Mol Biol Cell 8: 1805-1814).

On the other hand, we demonstrate here that INO1 mRNA can be upregulated in the presence of p180 in a UPR-independent fashion. Similarly, Stroobants et al showed that the IR is not necessarily linked to the UPR, as ire1Δ cells grown in the presence of oleate as the sole carbon source were capable of undergoing membrane proliferation upon overexpression of Hmg1p and the peroxisomal membrane protein, Pex15p (Stroobants et al. (1999) FEBS Lett 453: 210-214. (Others had shown that overexpression of HMG CoA-reductase (Hmg1p), which triggers the proliferation of karmellae, impaired the growth of ire1A cells, suggesting a block in membrane biogenesis, although membrane biogenesis per se was not assessed (Cox et al. (1997) Mol Biol Cell 8: 1805-1814)). They demonstrated that the level of INO1 mRNA was activated in response to growth on oleate, dependent upon the presence of oleate-specific transcriptional activators. The observation that there may be multiple mechanisms for activating INO1 is further substantiated by our observation that Ino2p, a basic helix-loop-helix transcription factor essential for the production of INO1 mRNA, is essential for the formation of p180-induced membranes. How INO2 is activated in response to p180 expression is unknown, however, it likely plays a critical role in the regulation of several phospholipid biosynthetic genes involved in the proliferation of intracellular membranes. Thus, it appears that the product of the INO1 gene, inositol-1-phosphate, is an important phospholipid component that is likely activated by a variety of mechanisms, Ire1p-dependent and independent, during conditions of ER stress.

Our observations that ΔCT-expressing cells failed to elicit a UPR and actually appeared to attenuate transcription of a UPR reporter gene suggest that the cellular response to p180 expression bypasses the UPR. One possible explanation for ΔCT-induced attenuation of transcription from the UPRE is that the Ire1p does not directly sense unfolded proteins, rather it senses and is held in an inactive state by levels of free Kar2p. Because ΔCT results in the upregulation of Kar2p through a mechanism distinct from the UPR, the additional free Kar2p may be available to maintain Ire1p in an inactive state. Evidence to support this postulate has recently been found in mammalian cells, in which the Kar2p homologue, BiP, binds the IRE1 protein and prevents its activation under normal conditions. Under conditions of ER stress, IRE1 is activated as BiP dissociates and binds to unfolded proteins (Bertolotti et al. (2000) Cell Biol 2: 326-332). If Ire1p activity is regulated by a similar mechanism in yeast, then increases in Kar2p induced by p180 could result in attenuation of UPR signaling.

Microsomes isolated from yeast cells expressing p180 constructs that contain the ribosome-binding domain (p180-FL and ACT) were shown to bind 2-4 times as many ribosomes as control microsomes in an in vitro ribosome-binding assay (Wanker et al. (1995) J Cell Biol 130: 29-39). This recruitment is thought to be mediated by interactions between the repeat regions of p180's ribosome-binding domain and the 28S ribosomal RNA (A. Savitz and D. Meyer, unpublished observations). The ability of p180 to recruit polysomes to the ER membrane could account for the presence of some of the normally cytosolically-translated mRNAs (such as PGK1) on the ER membrane (FIG. 20). Importantly, however, the overall level of PGK1 mRNA between p180-expressing and control cells remained unchanged. This suggests that mere recruitment of polysomes to the ER is not solely responsible for mRNA stabilization and that other factors are likely involved. Such factors could include targeting coupled with translation of mRNAs and/or protein translocation on the ER membrane.

Targeting of mRNA to the membrane in the presence of p180 influences its turnover. This was shown in experiments utilizing tetracycline-repressible forms of GFP and KAR2 signal sequence-modified GFP (FIG. 19) where mRNA turnover decreased about two to three fold. Another contributing factor would be translation on the membrane. This is supported by our data on SEC61 mRNA presented in FIG. 18 where turnover rates decreased by at least 8 fold and in other experiments by as much as 10 or 20 fold. Sec61p is a very hydrophobic pore-forming protein with multiple membrane-spanning domains. A previous study using a biological assay supports at least one of our interpretations. The investigators observed that the targeting the mRNA of a cytosolically-translated histone bearing a hydrophobic signal peptide prevented its otherwise rapid cell-cycle dependent degradation (Zambetti et al. (1990) J Cell Physiol 144: 175-182). This mislocalization of mRNAs to the ER presumably prevented their degradation by cytosolic enzymes.

In p180-expressing cells, membrane-localized mRNAs could achieve protection from degradation by their physical association with ribosomes that are tightly bound to the membrane. Another interesting possibility is that mRNAs that are targeted to the ER interact directly with p180. If such an interaction occurs, it may be with regions traditionally recognized to promote mRNA stability, such as the 3′UTR. Interestingly, Sabatini reported in 1975 that in WI-38 cells (a human fibroblast cell line with well-developed rough ER) mRNAs can remain associated with the ER membrane after the membranes have been stripped of ribosomes (Lande et al. (1975) J Cell Biol 65: 513-528). The poly-A portion of these mRNAs remains attached even following extensive treatment with RNase. An interesting and testable hypothesis is that p180 binds elements at or near the poly-A tail of certain mRNAs, thereby increasing their association with the membrane and protecting them from degradation. Lastly, p180 could be associating with both forms of RNA, ribosomal and messenger, stabilizing the translational complex. In this way an mRNA could experience increased levels of translation, surrounded by a variety of factors that render it inaccessible to enzymes involved in its turnover.

Thus far, these experiments do not address the role of p180 in mammalian secretory cells. It is still unknown whether the events that occur upon expression of a foreign membrane protein in yeast reproduce those that occur during terminal differentiation of cells with a high capacity for secretion. Our results, however, are promising. As the complexity and functionality of the membranes produced in response to p180 expression far exceeds that which is seen for other proteins, our studies assume a greater significance. The fact that mRNA stabilization is associated with p180 expression has generated a testable hypothesis as to how increases in secretory pathway proteins could occur in the terminal differentiation in a variety of tissues including pancreas, liver, mammary gland and plasma cells of the immune system.

Materials and Methods

Yeast strains

Saccharomyces cerevisiae strain SEY6210 (MATα:, leu2-3, ura3-52, his3-≢200, trp1-Δ901, lys2-801, suc2-D9) (Wilsbach et al. (1993) Embo J 12: 3049-3059) was used in the experiments involving SEC61-lacZ and membrane fractionation. Strain BY4733 (MATa, his3Δ200, leu2Δ0, met15Δ0, trp1Δ63, ura3Δ0) (Brachmann et al. (1998) Yeast 14: 115-132) was used for the generation of the tet-SEC61 and tet-GFP strains. Δire1 was created in the strain FY1679-18B (MATΔ; ura3-52, trp1-Δ63, leu2Δ1, his3Δ200, GAL2) (Winston et al. (1995) Yeast 11: 53-55) using PCR-based gene replacement. Primers consisting of 40 bases up and downstream of the IRE1 coding sequence and 20 bases identical to sequences in pRS303 were used to amplify the HIS3 gene. (5′ ATG CGT CTA CTT CGA AGA AAC ATG TTA GTA TTG ACA CTG Cga ttg tac tga gag tgc acc-3′ (SEQ ID NO:6) and 5′-TTA TGA ATA CAA AAA TTC ACG TAA AAT TTG ATC GTC ACT Tca tct gtg cgt cac acc g-3′ (SEQ ID NO:7)). Transformants were selected on media lacking histidine, confirmed to be devoid of IRE1 by PCR analysis, and individual colonies were tested for sensitivity to tunicamycin. The tet-repressible SEC61 strain was created by replacement of the region between CSR1 gene and the SEC61 gene with DNA between the CSR1 EcoRV site and SEC61 BamHI site from the pCM182 tet-SEC6J. Gene replacements were confirmed by PCR.

Growth conditions and Yeast Transformations

Yeast cells were grown in 4% dextrose, (Fisher Scientific), 0.17% Difco yeast nitrogen base without amino acids and ammonium sulfate (Fisher Scientific), and 5% ammonium sulfate (Fisher Scientific) with the addition of nucleotides and amino acids as appropriate. Transformations were performed using lithium acetate transformation procedures as described (Gietz et al. (1995) Yeast 11: 355-360). Induction of expression of p180 or its derivatives from pYEX-BX plasmids was carried out by growing the cells overnight to an O.D. of 0.5-3.5, diluting the cells to an O.D. of 0.5 and adding of CuSO₄ to 0.5 mM. Cells were induced for 5 to 7 hours. Where indicated, tunicamycin (Calbiochem) was added to 2 μg/ml for 2 hours, and doxycyline (Sigma) was added to 5 μg/ml.

Plasmids

Plasmids were constructed according to standard techniques. The p180 constructs (full-length, ACT, ANT, MA) in pYEX-BX have been described previously (Becker et al. (1999) J Cell Biol 146: 273-284. pBSK-TRP1 was a gift from Greg Payne (UCLA, Los Angeles, Calif.). The SEC61-LacZ plasmid encodes DNA from −1000 to +51 of the SEC61 gene, fused to the E. coli LacZ gene in the XhoI/XbaI sites of pRS314 (Sikorski and Hieter (1989) Genetics 122: 19-27). pMCZ-Y was a gift from K. Mori (HSP Research Institute, Shimogyo-ku, Japan). (Mori et al. (1996) Genes Cells 1: 803-817). pCM182 was a gift from E. Herrero (Universitat de Lleida, Spain) (Gari et al. (1997) Yeast 13: 837-848). The tet-SEC61 plasmid was created by PCR amplification of a 3′ truncation of the CSR1 gene followed by digestion with EcoRV (endogenous) and BglI (primer-encoded) and a BamHI/EcoRI digest of pBKS-TRP1 followed by simultaneous ligation of these DNA fragments into the EcoRI/EcoRV sites of pCM182. Amplification of the coding sequence for SEC61 was followed by digestion with BamHI (in primer) and StuI (endogenous) and cloning of this fragment into the same sites in pCM182.

Transcription Run-On Assay

Transcription run-ons were performed essentially as described (Parker et al. (1991) Methods Enzymol 194: 415-423), except that ATP was added to 6 mM and phosphocreatine and creatine phosphokinase were omitted. PCR products encoding the entire open reading frames of the SEC61, PYK1, and GFP genes and from nucleotides 1200-2048 of the KAR2 gene were gel purified using a Gel Extraction Kit (Qiagen), precipitated, and redissolved in ddH₂O. PCR products were denatured by the addition of NaOH to 125 mM and 1 μg of DNA was spotted on to Magna Membranes (Osmonics, Inc.). Radiolabeled transcripts were isolated as the supernatants from the protein precipitation step of the MasterPure genomic DNA isolation kit (Epicentre), and incubated overnight at 42 degrees with blots that had prehybridized in prehybridization/hybridization solution (Brown and MacKay (1997) Current Protocols in Mol. Biol.). Blots were rinsed twice, washed twice for 10 minutes at 42 degrees and twice for 30 minutes at 50 degrees in 0.1× SSC, 0.5% SDS and exposed PhosphorImaging screens.

Membrane Fractionation

Strains harboring the YEX vector alone or the ACT construct of p180 were induced in wild-type yeast for 6 hours with copper sulfate. Approximately 1000 OD₆₀₀ of each strain was harvested by centrifugation. The cells were ground with a mortar and pestle in liquid nitrogen and resuspended in 5 ml Buffer I (20 mM HEPES pH 7.4, 10 mM potassium acetate, 2 mM magnesium acetate). The fractionation was carried out as described (Stoltenburg et al. (1995) Biotechniques 18: 564-568), except RNA from the pellet containing the membrane-bound polysomes was released by extraction with glass beads in LETS buffer (0.1 M LiCl, 0.01 M EDTA, 0.01 M Tris/HCl pH 7.4, 0.2% SDS) instead of 0.2% sodium deoxycholate and 0.5% Tween-20. RNA was extracted as described below.

RNA Isolation and Northern Blot Analysis

For total cellular RNA extraction, RNA was isolated as a by-product of DNA using the MasterPure genomic DNA isolation kit (Epicentre, Madison, Wis.), and dissolved in formamide. For membrane fractionation experiments, RNA was extracted several times from free and membrane-bound polysome suspensions in an equal volume of DEPC H₂O-saturated phenol/choloform/iso-amylalcohol (49:49:2), precipitated overnight at −20° C. in isopropanol, and dissolved in formaldehyde. Five micrograms of RNA were loaded per lane for Northern blots, except for tet-SEC61 and tet-KAR2ssGFP blots, where 3 μg RNA were used. Gel electrophoresis, capillary transfer to Magna Membranes (Osmonics, Vista, Calif.) and probing and washing of the blots were performed as described (Brown and MacKay (1997) Current Protocols in Mol. Biol.). DNA fragments used as probes were generated from yeast genomic DNA or plasmid DNA with the following oligonucleotide primers: KAR2: 5′-AAC TGC AGA TGT TTT TCA ACA GAC TAA GCG C-3′ (SEQ ID NO:8) and 5′-ACG CAT GTC GAC CTA CAA TTC GTC GTG TTC GAA-3′ (SEQ ID NO:9), SEC61: 5′-CTA GCT GTC GAC ATG TCC TCC AAC CGT GTT CTA GAC T-3′ (SEQ ID NO:10) and 5′-AAC TGC AGT CAC ATC AAA TCA GAA AAT CCT GGA ACG-3′ (SEQ ID NO:11), INO1, 5′-CCT TGA TTT ATT CTG TTT C-3′ and 5′-ATC TCT CTT GGA ATC TTA GTT GG-3′ (SEQ ID NO:12), PGK1: 5′-AAC GTC CCA TTG GAC GGT AA-3′ (SEQ ID NO:13), and 5′-TCT TGT CAG CAA CCT TGG CA-3′ (SEQ ID NO:14), PYK1: 5′-CTA GCT GTC GAG ATG TCT AGA TTA GAA AGA TTG ACC TCA TTA AAC-3′ (SEQ ID NO:15) and 5′-AAC TGC AGT TAA ACG GTA GAG ACT TGC AAA GTG TTG-3′ (SEQ ID NO:16), GFP: 5′-GGG GAT CCC ATG CTC GAG AGT AAA GGA GAA GAA C-3′ (SEQ ID NO:17) and 5′-GTG TTT GTA TAG TTC ATC CAT GCC ATG-3′ (SEQ ID NO:18), PEP4: 5′-GCA TTA TTG CCA TTG GCC TT-3′ (SEQ ID NO:19) and 5′-GTG TCT TGA GAA ATG TAA CCT TCC A-3′ (SEQ ID NO:20). Radiolabeling was carried out using the Random Prime DNA labeling kit (Invitrogen Life Technologies, Inc., Carlsbad, Calif.) according to the manufacturer's instructions. Blots were exposed to PhosphorImaging screens, quantitated using ImageQuant software (Molecular Dynamics, Sunnyvale, Calif.). Values reported reflect normalization to levels of PYK1 or PGK1 RNA, except for membrane fractionation experiments, where RNA levels were normalized to scanned and quantified levels methylene blue-stained RNA.

β-Galactosidase Assays

Quantitation of β-galactosidase activity was performed using liquid assays with o-nitrophenyl-β-D-galactopyranoside (ONPG) as a substrate. Yeast transformants were grown overnight in appropriate synthetic media, then diluted to 0.5 O.D. and grown for 5 hours with 0.5 mM CuSO₄ to induce expression of p180 constructs. 10 ml cells were harvested, extracts prepared and enzyme activity quantitated as described (Breeden and Nasmyth (1987) Cell 48: 389-399) with the following modifications: Cells were lysed in Z-buffer without β-mercaptoethanol, and reactions carried out in a microtiter plate with 5 μl extract, 100 μZ buffer with 1 mM DTT in place of β-mercaptoethanol and 0.7 mg/ml ONPG. Change in absorbance at 430 nm was measured following addition of 30 μl 1M Na₂CO₃ to stop the reaction. Protein concentration was measured with the BioRad protein assay according to the manufacturer's instructions. Units are defined as 1000×A₄₃₀/(c×t×v) where c=protein concentration in mg/ml, t=time of reaction and v-volume of extract in

Electron Microscopy

Yeast cells were grown to mid-log phase in the presence of copper to induce expression of p180 constructs. Approximately 108 cells were harvested, washed once in 100 mM Tris-sulfate, pH 9.0, once in 100 mM Tris-sulfate, pH 9.0+10 mM DTT, and once in sorbitol buffer (1.2 M sorbitol; 20 mM potassium phosphate, pH 7.4; 5 mM DTT). Cells were resuspended in spheroplasting media (SD media+1.2 M sorbitol; 20 MM potassium phosphate, pH 7.4; 5 mM DTT) with 100 units oxalyticase and incubated at 45 degrees for 30 minutes. Cells were fixed in two steps: one 30 minute incubation at 4 degrees in 4% glutaraldehyde solution, (4% glutaraldehyde, 0.1 M cacodylic acid, 1.2 M sorbitol, 20 mM potassium phosphate, pH 7.4) and one 20 minute incubation at 4 degrees in 2% glutaraldehyde solution (0.375 M sodium cacodylate, 3.75% sucrose, 2% glutaraldehyde). Cells were washed three times in 0.5 M sodium cacodylate, 5% sucrose and resuspended in 2% glutaraldehyde solution. The remainder of the process was carried out as described (Block-Alper et al. (2002) Mol Biol Cell 13: 40-51.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of increasing the intracellular half-life of an mRNA in a eukaryotic cell, said method comprising
increasing the intracellular level of a ribosome receptor, as compared to the level of said ribosome receptor in an unmodified or untreated eukaryotic cell, whereby the increase in intracellular level of said ribosome receptor results in an increase in the half-life of said mRNA.

2. The method of claim 1, wherein said mRNA encodes a heterologous protein.

3. The method of claim 1, wherein said mRNA encodes a heterologous protein comprising a secretion signal.

4. The method of claim 1, wherein said mRNA encodes a membrane bound protein.

5. The method of claim 1, wherein said mRNA encodes a secreted protein.

6. The method of claim 1, wherein said mRNA is encoded by a nucleic acid construct that is not integrated into the genome of said cell.

7. The method of claim 1, wherein said mRNA is encoded by a nucleic acid construct that is integrated into the genome of said cell.

8. The method of claim 1, wherein said mRNA is an mRNA endogenous to said cell.

9. The method of claim 1, wherein expression of said mRNA is under control of a constitutive promoter.

10. The method of claim 1, wherein expression of said mRNA is under control of an inducible promoter.

11. The method of claim 1, wherein said eukaryotic cell is a cell selected from they group consisting of a fungal cell, a plant cell, a vertebrate cell, a protozoan cell, and an invertebrate cell.

12. The method of claim 1, wherein said eukaryotic cell is a cell selected from they group consisting of a fungal cell, a plant cell, an insect cell, and a mammalian cell.

13. The method of claim 1, wherein the eukaryotic cell is a mammalian cell.

14. The method of claim 1, wherein the eukaryotic cell is a human cell.

15. The method of claim 1, wherein the eukaryotic cell is a CHO cell.

16. The method of claim 1, wherein said increasing the intracellular level of a ribosome receptor comprises transfecting said cell with a nucleic acid construct encoding said ribosome receptor.

17. The method of claim 1, wherein said increasing the intracellular level of a ribosome receptor comprises modifying or replacing the endogenous promoter regulating expression of an endogenous ribosome receptor.

18. The method of claim 16, wherein said transfecting produces a stable transfection.

19. The method of claim 16, wherein said transfecting produces a transient transfection.

20. The method of claim 16, wherein nucleic acid construct comprises a plurality of regions each encoding a ribosome receptor.

21. The method of claim 1, wherein said ribosome receptor is a mammalian ribosome receptor.

22. The method of claim 1, wherein said ribosome receptor is a full-length ribosome receptor.

23. A method of increasing production of a protein by a eukaryotic cell, said method comprising:
increasing the intracellular half-life of an mRNA encoding said protein by increasing the intracellular level of a ribosome receptor in said cell, as compared to the level of said ribosome receptor in an unmodified or untreated eukaryotic cell of the same type, whereby the increase in intracellular level of said ribosome receptor results in an increase in the half-life of said mRNA and resulting in an increase in expression of said protein.

24. The method of claim 22, wherein said mRNA encodes a heterologous protein.

25. The method of claim 22, wherein said mRNA encodes a heterologous protein comprising a secretion signal.

26. The method of claim 22, wherein said mRNA encodes a membrane bound protein.

27. The method of claim 22, wherein said mRNA encodes a secreted protein.

28. The method of claim 22, wherein said mRNA is encoded by a nucleic acid construct that is not integrated into the genome of said cell.

29. The method of claim 22, wherein said mRNA is encoded by a nucleic acid construct that is integrated into the genome of said cell.

30. The method of claim 22, wherein said mRNA is an mRNA endogenous to said cell.

31. The method of claim 22, wherein expression of said mRNA is under control of a constitutive promoter.

32. The method of claim 22, wherein expression of said mRNA is under control of an inducible promoter.

33. The method of claim 22, wherein said eukaryotic cell is a cell selected from they group consisting of a fungal cell, a plant cell, a vertebrate cell, a protozoan cell, and an invertebrate cell.

34. The method of claim 22, wherein said eukaryotic cell is a cell selected from they group consisting of a fungal cell, a plant cell, an insect cell, and a mammalian cell.

35. The method of claim 22, wherein the eukaryotic cell is a mammalian cell.

36. The method of claim 22, wherein the eukaryotic cell is a human cell.

37. The method of claim 22, wherein the eukaryotic cell is a CHO cell.

38. The method of claim 22, wherein said increasing the intracellular level of a ribosome receptor comprises transfecting said cell with a nucleic acid construct encoding said ribosome receptor.

39. The method of claim 22, wherein said increasing the intracellular level of a ribosome receptor comprises modifying or replacing the endogenous promoter regulating expression of an endogenous ribosome receptor.

40. The method of claim 38, wherein said transfecting produces a stable transfection.

41. The method of claim 38, wherein said transfecting produces a transient transfection.

42. The method of claim 38, wherein nucleic acid construct comprises a plurality of regions each encoding a ribosome receptor.

43. The method of claim 22, wherein said ribosome receptor is a mammalian ribosome receptor.

44. The method of claim 22, wherein said ribosome receptor is a full-length ribosome receptor.

45. The method of claim 22, further comprising maintaining said cell under cell culture conditions to secrete said recombinant protein therefrom.

46. The method of claim 22, further comprising purifying the protein secreted from said cell.

47. A kit for increasing the half-life of an mRNA in a eukaryotic cell, said kit comprising:
a container containing a nucleic acid construct encoding one or more ribosome receptors; and
instructional materials teaching that increasing the intracellular level of a ribosome receptor will increase the intracellular half-life of an mRNA.

48. The kit of claim 47, further comprising a vector for introducing a nucleic acid encoding said mRNA into said cell.

49. The kit of claim 47, further comprising a transfection reagent for introducing said nucleic acid construct into said cell.

50. A kit for increasing the half-life of an mRNA in a eukaryotic cell, said kit comprising:
a container containing a eukaryotic cell that has been treated or modified to express an elevated level of ribosome receptor as compared to an untreated or unmodified cell of the same type; and
instructional materials teaching that increasing the intracellular level of a ribosome receptor will increase the intracellular half-life of an mRNA.

51. The kit of claim 50, further comprising a vector for introducing a nucleic acid encoding said mRNA into said cell.

52. The kit of claim 50, further comprising a transfection reagent for introducing said nucleic acid construct into said cell.
